SEQUENCE LISTING (19 sequences)

SEQ ID NO: 1 - protein, 334 aa, Homo sapiens
  misc_feature: Incyte ID No: 347021CD1
MNLSLVLAAFCLGIASAVPKFDQNLDTKWYQWKATHRRLYGANEEGWRRAVWEKNMKMIELHNGEYSQGKLGFTMAMNAFGDMTNEEFRQMMGCFRNQKFRKGKVFREPLFLDLPKSVDWRKKGYVTPVKNQKQCGSCWAFSATGALEGQMFRKTGKLVSLSEQNLVDCSRPQGNQGCNGGFMARAFQYVKENGGLDSEESYPYVAVDEICKYRPENSVANDTGFTMVAPGKEKALMKAVATVGPISVAMDAGHSSFQFYKSGIYFEPDCSSKNLDHGVLVVGYGFEGANSNNSKYWLVKNSWGPEWGSNGYVKIAKDKNNHCGIATAASYPNV

SEQ ID NO: 2 - DNA, 1366 bp, Homo sapiens
  misc_feature: Incyte ID No: 347021CB1
ctcagaggcttgtttgctgagggtgcctgcgcagctgcgacggctgctggttttgaaacatgaatctttcgctcgtcctggctgccttttgcttgggaatagcctccgctgttccaaaatttgaccaaaatttggatacaaagtggtaccagtggaaggcaacacacagaagattatatggcgcgaatgaagaaggatggaggagagcagtgtgggaaaagaatatgaaaatgattgaactgcacaatggggaatacagccaagggaaacttggcttcacaatggccatgaatgcttttggtgacatgaccaatgaagaattcaggcagatgatgggttgctttcgaaaccagaaattcaggaaggggaaagtgttccgtgagcctctgtttcttgatcttcccaaatctgtggattggagaaagaaaggctacgtgacgccagtgaagaatcagaaacagtgtggttcttgttgggcttttagtgcgactggtgctcttgaaggacagatgttccggaaaactgggaaacttgtctcactgagcgagcagaatctggtggactgttcgcgtcctcaaggcaatcagggctgcaatggtggcttcatggctagggccttccagtatgtcaaggagaacggaggcctggactctgaggaatcctatccatatgtagcagtggatgaaatctgtaagtacagacctgagaattctgttgctaatgacactggcttcacaatggtcgcacctggaaaggagaaggccctgatgaaagcagtcgcaactgtggggcccatctccgttgctatggatgcaggccattcgtccttccagttctacaaatcaggcatttattttgaaccagactgcagcagcaaaaacctggatcatggtgttctggtggttggctacggctttgaaggagcaaattcgaataacagcaagtattggctcgtcaaaaacagctggggtccagaatggggctcgaatggctatgtaaaaatagccaaagacaagaacaaccactgtggaatcgccacagcagccagctaccccaatgtgtgagctgatggatggtgaggaggaaggacttaaggacagcatgtctggggaaattttatcttgaaactgaccaaacgcttattgtgtaagataaaccagttgaatcatggaggatccaagttgagattttaattctgtgacatttttacaagggtaaaatgttaccactactttaattattgttatacacagctttatgatatcaaagactcattgcttaattctaagacttttgaattttcattttttaaaaagatgtacaaaacagtttgaaataaattttaattcgtatataaaaaaaaaaa

SEQ ID NO: 3 - DNA, 219 bp, Homo sapiens
  misc_feature: Incyte ID No: 347021H1
cagatgttccggaaaactgggcaacttgtctcactgagcgagcagaatctggtggactgttcgcgtcctcaaggcaatcagggctgcaatggtggcttcatggctagggccttccagtatgtcaaggagaacggaggcctggactctgaggaatcctatccatatgtagcagtggatgaaatctgtaagtacagacctgagaattctgttgctaatgac

SEQ ID NO: 4 - DNA, 512 bp, Homo sapiens
  misc_feature: Incyte ID No: 347021R1
  unsure 494: a, t, c, g, or other
cagatgttccggaaaactgggaaacttgtctcactgagcgagcagaatctggtggactgttcgcgtcctcaaggcaatcagggctgcaatggtggcttcatggctagggccttccagtatgtcaaggagaacggaggcctggactctgaggaatcctatccatatgtagcagtggatgaaatctgtaagtacagacctgagaattctgttgctaatgacactggcttcacagtggtcgcacctggaaaggagaaggccctgatgaaagcagtcgcaactgtggggcccatctccgttgctatggatgcaggccattcgtccttccagttctacaaatcaggcatttattttgaaccagactgcagcagcaaaaacctggatcatggtgttctggtggttggctacggctttgaaggagcaaattcgaataacagcaagtatttggctcgtcaaaaacagcttggggtccagaattggggcttcgaatggctatntaaaaattgcccaaagac

SEQ ID NO: 5 - DNA, 210 bp, Homo sapiens
  misc_feature: Incyte ID No: 389479H1
cttccagtatgtcaaggagaacggaggcctggactctaaggaatcctatccatatgtagcagtggatgaaatctgtaagtacagacctgagaattctgttgctaatgacactggcttcacagtggtcgcacctggaaaggagaaggccctgatgaaagcagtcgcaactgtggggcccatctccgttgctatggatgcaggccattcgtc

SEQ ID NO: 6 - DNA, 474 bp, Homo sapiens
  misc_feature: Incyte ID No: 389479T6
tttgtacatctttttaaaaaatgaaaattcaaaagtcttagaattaagcaatgagtctttgatatcataaagctgtgtataacaataattaaagtagtggtaacattttacccttgtaaaaatgtcacagaattaaaatctcaacttggatcctccatgattcaactggtttatcttacacaataagcgtttggtcagtttcaagataaaatttccccagacatgctgtccttaagtccttcctcctcaccatccatcagctcacacattggggtagctggctgctgtggcgattccacagtggttgttcttgtctttggctatttttacatagccattcgagccccattctggaccccagctgtttttgacgagccaatacttgctgtattcgaatttgctccttcaaagccgtagccaaccaccagaacaccatgatccaggtttttgctggctgcagtctggttcaaaata

SEQ ID NO: 7 - DNA, 232 bp, Homo sapiens
  misc_feature: Incyte ID No: 2554720H1
ctcagaggcttgtttgctgagggtgcctgcgcactgcgacggctgctggttttgaaacatgaatctttcgctcgtcctggctgccttttgcttgggaatagcctcccgtgttccaaaatttgaccaaaatttggatacaaagtggtaccagtggaaggcaacacacagaagattatatggcgcgaatgaagaaggatggaggagagcagtgtgggaaaagaatatgaaaatg

SEQ ID NO: 8 - DNA, 1342 bp, Homo sapiens
  misc_feature: Incyte ID No: 2554720CA2
ctcagaggcttgtttgctgagggtgcctgcgcagctgcgacggctgctggttttgaaacatgaatctttcgctcgtcctggctgccttttgcttgggaatagcctccgctgttccaaaatttgaccaaaatttggatacaaagtggtaccagtggaaggcaacacacagaagattatatggcgcgaatgaagaaggatggaggagagcagtgtgggaaaagaatatgaaaatgattgaactgcacaatggggaatacagccaagggaaacatggcttcacaatggccatgaatgcttttggtgacatgaccaatgaagaattcaggcagatgatgggttgctttcgaaaccagaaattcaggaaggggaaagtgttccgtgagcctctgtttcttgatcttcccaaatctgtggattggagaaagaaaggctacgtgacgccagtgaagaatcagaaacagtgtggttcttgttgggcttttagtgcgactggtgctcttgaaggacagatgttccggaaaactgggaaacttgtctcactgagcgagcagaatctggtggactgttcgcgtcctcaaggcaatcagggctgcaatggtggcttcatggctagggccttccagtatgtcaaggagaacggaggcctggactctgaggaatcctatccatatgtagcagtggatgaaatctgtaagtacagacctgagaattctgttgctaatgacactggcttcacagtggtcgcacctggaaaggagaaggccctgatgaaagcagtcgcaactgtggggcccatctccgttgctatggatgcaggccattcgtccttccagttctacaaatcaggcatttattttgaaccagactgcagcagcaaaaacctggatcatggtgttctggtggttggctacggctttgaaggagcaaattcgaataacagcaagtattggctcgtcaaaaacagctggggtccagaatggggctcgaatggctatgtaaaaatagccaaagacaagaacaaccactgtggaatcgccacagcagccagctaccccaatgtgtgagctgatggatggtgaggaggaaggacttaaggacagcatgtctggggaaattttatcttgaaactgaccaaacgcttattgtgtaagataaaccagttgaatcattgaggatccaagttgagattttaattctgtgacatttttacaagggtaaactctctccccctactttaattacttgttatacaccagctcttatgatatccaagactcatttgcttaattctcaaacaaaaaaacaaaggcgcccctgactataacttgcacacatgactcac

SEQ ID NO: 9 - DNA, 102 bp, Homo sapiens
  misc_feature: Incyte ID No: 2555589F6
ctttatgatatcaaagactcattgcttaattctaagacttttgaattttcattttttaaaaagatgtacaaaacagtttgaaataaattttaattcgtatat

SEQ ID NO: 10 - DNA, 1807 bp, Rattus norvegicus
  misc_feature: Incyte ID No: 223055_Rn.10
gcgggccttgccggggccgcagcctgagagcctttaaagccggagccccgcgctgcttttccagattctcggacctcggcgacctccggggatccgagtttgcagactacgtgtgtgcgcagctagccacctcaggtgtttgaaccatgacccctttactcctcctggctgtcctctgcttgggaacagccttagccactccaaaatttgatcaaacgtttaatgcacagtggcaccagtggaagtccacacacagaagactgtatggcacgaatgaggaagagtggaggagagcagtgtgggagaagaacatgaggatgatccagctacacaatggggagtacagcaacgggaagcacggctttaccatggagatgaacgccttcggtgacatgaccaatgaggaattcaggcagatagtgaatggctatcgccaccagaagcacaagaagggaaggttatttcaggaacctctgatgctgcagatccccaagactgtggactggagagaaaagggttgtgtgactcctgtgaagaatcagggccagtgtggttcttgctgggcttttagcgcatcgggttgcctagaaggacagatgttccttaagactggcaaactgatctcactgagtgaacagaaccttgtggactgttctcacgatcaaggcaatcagggctgtaatggaggcctgatggattttgctttccagtacattaaggaaaatggaggtctggactcagaggagtcttatccctatgaagcaaaggatggatcttgtaaatacagagctgagtatgctgtggctaacgacacagggtttgtggatatccctcagcaagagaaagccctcatgaaggctgtagcgacggtggggcctatttctgttgccatggatgcaagccatccgtctctccagttctatagttcaggtatctactatgaacccaactgtagcagcaaggacctcgaccatggggttctggtggttggctatggttatgaaggaacagattcaaataaggataaatactggcttgtcaaaaacagctggggtaaagaatggggtatggatggctacatcaaaatagccaaagaccggaacaaccactgcggacttgccaccgcagccagctatcctatcgtgaattgatggacagcgataataaggacttacggacactacatccgaaggagttcatcttaaaactgaccaaacccgtctctgagtgagaccatggtacttgaatcgttcaggatccaagtcacgatttaaattctgttgacatttttacatgggttaaatgttaccactacttaaaactcctgttataaacagctttataatattggacacttaatgcttaattctgattctggaatatttgttttataaaagttgtataaaactttctttaccttttaaaaataaattttagctcagtgcatgtgtgtgtgtatgggttaggggaacttcctgtgtgaaatgtgttcacaaatgtttgagactaaagactgactgattccagatgtccggactgattcgggtgtcagtggtagacctggggaaaggtgacaggtgctctggatggagccttctgattttacctcagcgtcctgtcaggttaggtatgtgtaagtaaatctagcttatggggtaattgttttttctttatttgtgtgagtatgtgtgtgtggaggtcagagaacaactcatttctacagtgtgttgatcctagcgatcaaaatcaggttgttaggctggaccacaggtgccttttactactgatgtatcttgccgccccactg

SEQ ID NO: 11 - DNA, 699 bp, Rattus norvegicus
  misc_feature: Incyte ID No: 703912760J1
ctcagctctgtatttacaagatccatcctttgcttcatagggataagactcctctgagtccagacctccattttccttaatgtactggaaagcaaaatccatcaggcctccattacagccctgattgccttgatcgtgagaacagtccacaaggttctgttcactcagtgagatcagtttgccagtcttaaggaacatctgtccttctaggcaacccgatgcgctaaaagcccagcaagaaccacactggccctgattcttcacaggagtcacacaacccttttctctccagtccacagtcttggggatctgcagcatcagaggttcctgaaataaccttcccttcttgtgcttctggtggcgatagccattcactatctgcctgaattcctcattggtcatgtcaccgaaggcgttcatctccatggtaaagccgtgcttcccgttgctgtactccccattgtgtagctggatcatcctcatgttcttctcccacactgctctcctccactcttcctcattcgtgccatacagtcttctgtgtgtggacttccactggtgccactgtgcattaaacgtttgatcaaattttggagtggctaaggctgttcccaagcagaggacagccaggaggagtaaaggggtcatggttcaaacacctgaggtggctagctgcgcaccacacgtaggtctgcaaaa

SEQ ID NO: 12 - DNA, 705 bp, Macaca fascicularis
  misc_feature: Incyte ID No: 003170_Mf.1
tgttgccgacaatggaggcctggactctgaggaatcctatccatatgaggcaacagaagaatcctgtaagtacaatcccgagtattctgttgctaatgacaccggctttgtggacatccctaagcaggagaaggccctgatgaaggcagttgcaactgtggggcccatttctgttgctattgatgcaggtcatgagtccttcatgttctataaagaaggcatttattttgagccagactgtagcagtgaagacatggatcatggtgtgctggtggttggctatggatttgaaagcacagaatcagacaacagtaaatattggctggtgaagaacagctggggtgaagaatggggcatgggtggctacataaagatggccaaggaccggagaaaccattgtggaattgcctcagcagccagctaccccactgtgtgagctggtggacagtgatgaggaaggacttgactggggatggcgcatgcatgggaggaattcatcttcagtctaccagcccctgctgtgtcggatacacactcgaatcattgaagatccaagtgtgatttgaattctgtgatattttcacactggtaaatgttacctctattttaattactgctataaataggtttatattattgattcacttactgactttgcatttttgtttttaaaaggatgtataaatttttacctgtttaaataaa

SEQ ID NO: 13 - DNA, 1328 bp, Canis familiaris
  misc_feature: Incyte ID No: 007499_Cf.1
gctaagtcgacgctgccgggatcccgtttttgaaacatgaatccttcactcttcttgactgccctttgcttgggaatagcctcagcagctcccaaatttgatcaaagcttaaatgcacagtggtaccagtggaaggcaacgcacaggagattatatggcatgaatgaagaaggatggaggagagcagtgtgggagaagaatatgaaaatgattgaactgcataatcgggaatacagccaagggaaacatggcttcacaatggcaatgaatgcctttggtgacatgaccaatgaagaattcaggcaggtgatgaatggctttcaaaaccagaagcacaagaaagggaaaatgttccaagaacctctctttgctgagatccccaaatcagtggactggagagagaaaggctatgtaactcctgtgaagaatcagggtcagtgtggttcttgttgggcttttagtgcaactggtgcccttgaaggacagatgttccggaaaacgggcaaacttgtgtcactgagtgagcaaaacctggtggactgctctagggctcaaggtaacgagggctgcaatggtggcctgatggataacgccttccggtatgttaaggacaatggaggcctggactcagaggaatcttatccgtatcttggaagggacacagagacctgcaattacaagcctgagtgttctgctgccaatgacactggctttgtggacctccctcaacgggagaaggccctaatgaaagcagtggcaactctagggcccatctctgttgctattgatgcaggccatcagtctttccaattctacaaatcaggcatttattttgatccagactgcagcagcaaagatctagatcatggtgttctggtggttggctatggctttgaaggaacagattcaaataataaattttggattgtcaagaacagttggggtccagaatggggctggaatggctatgtaaaaatggccaaagaccagaacaaccactgtggaattgccacagcagccagctatcccactgtgtgagctgatggatcgcaagattagaggacttgaaggcagcatatctggaagaattttatcttaaagctgaccagactcttattgtataagataagacacttgaatcattgaggatccaagttgtgatttgaattctgtgacatttttatcagggtaaaacattaccattactttaattactgttatatatggctttataacgttgaagactcattacttaattctaagacttttgacttctcattttttcaaaagatatataaaactttgccttttgaaataaattttaattc

SEQ ID NO: 14 - DNA, 805 bp, Canis familiaris
  misc_feature: Incyte ID No: 704138617J1
gcgccgcaggtttttgaaacatgaatccttcactcttcttgactgccctttgcttgggaatagcctcagcagctcccaaatttgatcaaagcttaaatgcacagtggtaccagtggaaggcaacgcacaggagattatatggcatgaatgaagaaggatggaggagagcagtgtgggagaagaatatgaaaatgattgaactgcataatcgggaatacagccaagggaaacatggcttcacaatggcaatgaatgcctttggtgacatgaccaatgaagaattcaggcaggtgatgaatggctttcaaaaccagaagcacaagaaagggaaaatgttccaagaacctctctttgctgagatccccaaatcagtggactggagagagaaaggctatgtaactcctgtgaagaatcagggtcagtgtggttcttgttgggcttttagtgcaactggtgcccttgaaggacagatgttccggaaaacgggcaaacttgtgtcactgagtgagcaaaacctggtggactgctctagggctcaaggtaacgagggctgcaatggtggcctgatggataacgccttccggtatgttaaggacaatggaggcctggactcagaggaatcttatccgtatcttggaagggacacagagacctgcaattacaagcctgagtgttctgctgccaatgacactggctttgtggacctccctcaacgggagaaggccctaatgaaagcagtggcaactctaaggcccatctctgttgctattgatgcagagccatcagtcttttccaattctacaaa

SEQ ID NO: 15 - DNA, 846 bp, Canis familiaris
  misc_feature: Incyte ID No: 703985027J1
gaattaaaatttatttcaaaaggcaaagttttatatcttttgaaaaaatgagaagtcaaaagtcttagaattaagtaatgagtcttcaacgttataaagccatatataacagtaattaaagtaatggtaatgttttaccctgataaaaatgtcacagaattcaaatcacaacttggatcctcaatgattcaagtgtcttatcttatacaataagagtctggtcagctttaagataaaattcctccagatatgctgccttcaagtcctctaatcttgcgatccatcagctcacacagtgggatagctggctgctgtggcaattccacagtggttgttctggtctttggccatttttacatagccattccagccccattctggaccccaactgttcttgacaatccaaaatttattatttgaatctgttccttcaaagccatagccaaccaccagaacaccatgatctagatctttgctgctgcagtctggatcaaaataaatgcctgatttgtagaattggaaagactgatggcctgcatcaatagcaacagagatgggccctagagttgccactgctttcattagggccttctcccgttgagggaggtccacaaagccagtgtcattggcagcagaacactcaggcttgtaattgcaggtctctgtgtcccttccaagatacggataagattcctctgagtcccaggcctccattgtccttaacataccgggaaggcggttatccattcagagcaccattgcaggcctcgttaccttgagcctaagaagagtccaccaggtttgtccactcagtgacacaagtttcccgttttccggaacatttgt

SEQ ID NO: 16 - DNA, 1961 bp, Mus musculus
  misc_feature: Incyte ID No: 085657_Mm.1
tgggcggcgacctccggggatccgagtttgcagacttcttgtgcgcagctagccgcctcaggtgtttgaaccatgaatcttttactccttttggctgtcctctgcttgggaacagccttagctactccaaaatttgatcaaacctttagtgcagagtggcaccagtggaagtccacacacagaagactgtatggcacgaatgaggaagagtggaggagagcgatatgggagaagaacatgagaatgatccagctacacaacggggaatacagcaacgggcagcacggcttttccatggagatgaacgccttcggtgacatgaccaatgaggaattcaggcaggtggtgaatggctaccgccaccagaagcacaagaaggggaggctttttcaggaaccgctgatgcttaagatccccaagtctgtggactggagagaaaagggttgtgtgactcctgtgaagaaccagggccagtgcgggtcttgttgggcgtttagcgcatcgggttgcctagaaggacagatgttccttaagaccggcaaactgatctcactgagtgaacagaaccttgtggactgttctcacgctcaaggcaatcagggctgtaacggaggcctgatggattttgctttccagtacattaaggaaaatggaggtctggactcggaggagtcttaccccctatgaagcgaaggacggatcttgtaaatacagagccgagttcgctgtggctaatgacacagggttcgtggatatccctcagcaagagaaagccctcatgaaggctgtggcgactgtggggcctatttctgttgctatggacgcaagccatccgtctctccagttctatagttcaggcatctactatgaacccaactgtagcagcaagaacctcgaccatggggttctgttggtgggctatggctatgaaggaacagattcaaataagaataaatattggcttgtcaagaacagctggggaagtgaatggggtatggaaggctacatcaaaatagccaaagaccgggacaaccactgtggacttgccaccgcggccagctatcctgtcgtgaattgatgggtagcggtaatgaggacttatggacactatgtccaaaggaattcagcttaaaactgaccaaacccttattgagtcaaaccatggtacttgaatcattgaggatccaagtcatgatttgaattctgttgccatttttacatgggttaaatgttaccactacttaaaactcctgttataaacagctttataatattgaaaacttagtgcttaattctgagtctggaatatttgttttatataaaggttgtataaaactttctttacctcttaaaaataaattttagctcagtgtgggggatgggggcttcaggtgtgtatgtggtggcaaatgttcgggactaatactgatttcagggtaacatggtacagcaagaaaaaggtggcaggtgctctggagccttcggatttcacctcagtgtcccatcagattaggtatgtgttagtgaatctagcttgggaaaagtgttttctttttgtgtgtgtacatggtaaatgtgtggagttcagagaacaactcagttccacagtgttggtcctatcaggtcctagccatcaaaataaggttgtcaggctgggccacaggtgccttttactactgagctatcttgccagccccactagtttttaagtgacataattacgtatgtttatactataatgtgtttttgatatgatgtgtatactacaaaatgatctgatagggctaataagcttacctcaaatatttaattactttgttgaacttttctagctgtttttaaaatacacagtatattattgttggaaactgtcctgttatgtagaccaggctggccttaagcccatgatctcccttcctcagccttctaagggctaagataaacatgtaatgttataccct

SEQ ID NO: 17 - DNA, 257 bp, Mus musculus
  misc_feature: Incyte ID No: 701326170H1
gaacgcctttggagacatgaccagtgaagaattcaggcaggtgatgaatggctttcaaaaccgtaagcccaggaaggggaaagtgttccaggaacctctgttttatgaggcccccagatctgtggattggagagagaaaggctacgtgactcctgtgaagaatcagggtcagtgtggttcttgttgggcttttagtgctactggtgctcttgaaggacagatgttccggaaaactgggaggcttatctcactgagtg

SEQ ID NO: 18 - protein, 333 aa, Homo sapiens [sequence omitted]

SEQ ID NO: 19 - protein, 334 aa, Sus scrofa
  ref <308>: DATABASE ACCESSION NUMBER: Genbank ID No: g1468964
MKPSLFLTALCLGIASAAPKLDQNLDADWYKWKATHGRLYGMNEEGWRRAVWEKNMKMIELHNQEYSQGKHGFSMAMNAFGDMTNEEFRQVMNGFQNQKHKKGKVFHESLVLEVPKSVDWREKGYVTAVKNQGQCGSCWAFSATGALEGQMFRKTGKLVSLSEQNLVDCSRPQGNQGCNGGLMDNAFQYVKDNGGLDTEESYPYLGRETNSCTYKPECSAANDTGFVDIPQREKALMKAVATVGPISVAIDAGHSSFQFYKSGIYYDPDCSSKDLDHGVLVVGYGFEGTDSNSSKFWIVKNSWGPEWGWNGYVKMAKDQNNHCGISTAASYPTV

(19) United States
(12) Patent Application Publication
(10) Pub. No.: US 2003/0036102 A1
(43) Pub. Date: Feb. 20, 2003

(54) Cathepsin differentially expressed in lung cancer

(76) Inventors: Olga Bandman, Mountain View, CA (US); Purvi Shah, San Jose, CA (US); Paula K. Rickert, Pacifica, CA (US)

Correspondence Address: INCYTE GENOMICS, INC., LEGAL DEPARTMENT, 3160 PORTER DRIVE, PALO ALTO, CA, 94304, US

(21) Appl. No.: 10/143,650

(22) Filed: May 8, 2002

Related U.S. Application Data
(63) Continuation-in-part of application No. 09/519,283, filed on Mar. 7, 2000.
Application No. 09/519,283 is a division of application No. 08/883,526, filed on Jun. 26, 1997, now Pat. No. 6,033,893.

(51) Int. Cl. G01N 33/574; C07H 21/04; C12N 9/00; C12P 21/02; C12N 5/06

(52) U.S. Cl. 435/7.23; 435/69.1; 435/183; 435/320.1; 435/325; 536/23.2

(57) ABSTRACT

The invention is a new human cathepsin (LCAP), the cDNAs that encode LCAP, and antibodies that specifically bind LCAP that are used in methods for diagnosing and treating disorders of cell proliferation, particularly lung cancer, as associated with expression of LCAP. The invention provides expression vectors, host cells, and methods for making LCAP and agonists, antibodies and antagonists that specifically bind the protein.

This application is a continuation-in-part of U.S. Ser. No. 09/519,283, filed Mar. 7, 2000, which is a divisional of U.S. Pat. No. 6,033,893, which issued Mar. 7, 2000 and matured from U.S. Ser. No. 08/883,526, filed Jun. 26, 1997.

### FIELD OF THE INVENTION

This invention relates to a human cathepsin, its encoding cDNA, and an antibody which specifically binds the cathepsin and to their use to diagnose, to stage, to treat, or to monitor the progression of disorders of cell proliferation, particularly lung cancer and complications of lung cancer.

### BACKGROUND OF THE INVENTION

Cathepsins are a family of proteases which include the cysteine protease cathepsins B, H, K, L, and S. These enzymes have a role in processes that involve proteolysis and turnover of specific proteins and tissues in local microenvironments. Cathepsins also initiate proteolytic cascades by proenzyme activation, participate in the expression of functional MHC class II molecules which bind antigenic peptides, and process antigen in antigen-presenting cells. The various members of this family are differentially expressed, and cathepsin L is closely associated with monocytes, macrophages, and other cells of the immune system. The secreted forms of several members of this family function in tissue remodeling through degradation of collagen, laminin, elastin, and other structural proteins and are implicated in inflammation associated with immunological response and in metastasis (Huisman et al. (1974) Biochem Biophys Acta 370:297-307; Mizuochi (1994) Immunol Lett 43:189-193; and Baldwin (1993) Proc Natl Acad Sci 90:6796-6800).

The various cathepsin proteases differ in their gene structures and in their transcriptional regulation. The cathepsin L gene promoter has no TATA box but includes several SP-1 sites, two AP-2 transcription regulatory element binding sites, and a cAMP response element. Experimental data confirm that expression of cathepsin L is induced by malignant transformation, growth factors, tumor promoters, and cyclic AMP (Troen et al. (1991) Cell Growth Differ 2:23-31).

Abnormal regulation and expression of cathepsins is evident in various inflammatory disease states. In cells isolated from inflamed synovia, the mRNA for stromelysin, cytokines, TIMP-1, cathepsin, gelatinase, and other molecules is preferentially expressed. Expression of cathepsins L and D is elevated in synovial tissues from patients with rheumatoid arthritis and osteoarthritis. Cathepsin L expression may also contribute to the influx of mononuclear cells which exacerbates the destruction of the rheumatoid synovium (Keyszer (1995) Arthritis Rheum. 38:976-984).

The cathepsins are implicated in several other immune responses. In a rat model of human glomerular disease, the administration of a specific, irreversible inhibitor of cysteine protease (trans-epoxysuccinyl-L-leucylamido-(3-methyl)butane) significantly reduced proteinuria (Baricos (1991) Arch Biochem Biophys 288:468-72). The platelet aggregating cysteine protease implicated in thrombotic thrombocytopenic purpura shows the characteristics of a lysosomal cathepsin (Consonni (1994) Br J Hematol 87:321-324). In addition, the increased expression and differential regulation of the cathepsins is linked to the metastatic potential of a variety of cancers and as such is of therapeutic and prognostic interest (Chambers et al. (1993) Crit Rev Oncog 4:95-114).

The new human cathepsin, its encoding cDNA, and an antibody which specifically binds the cathepsin are useful to diagnose, to stage, to treat, or to monitor the progression or treatment of disorders of cell proliferation, particularly lung cancer and complications of lung cancer.

### SUMMARY OF THE INVENTION

The invention is a purified human cathepsin that has been designated LCAP, its encoding cDNA, and an antibody which specifically binds LCAP, each of which is useful to diagnose, to stage, to treat, or to monitor the progression or treatment of disorders of cell proliferation, particularly lung cancer and complications of lung cancer.

The invention provides an isolated cDNA comprising a polynucleotide encoding a protein having the amino acid sequence of SEQ ID NO:1. The invention also provides an isolated cDNA comprising a polynuclotide selected from a nucleic acid sequence of SEQ ID NO:2, a fragment of SEQ ID NO:2, a probe selected from SEQ ID NOs:3-9, an oligonucleotide extending from about nucleotide1011 to nucleotide 1086 of SEQ ID NO:2, a variant of SEQ ID NO:2 selected from the group consisting of SEQ ID NOs:10-17, array elements selected from SEQ ID NOs:2-17, and a substrate upon which cDNA is immobilized.

The invention provides a vector containing the cDNA encoding LCAP, a host cell containing the vector and a method for using the cDNA to make the protein, the method comprising culturing the host cell containing the vector containing the cDNA encoding the protein under conditions for expression and recovering the protein from the host cell culture. The invention also provides a transgenic cell line or organism comprising the vector containing the cDNA encoding LCAP. The invention further provides a composition, a substrate or a probe comprising the cDNA, a fragment, a variant, or complements thereof, which can be used in methods of detection, screening, and purification. In one aspect, the probe is a single-stranded complementary RNA or DNA molecule.

The invention provides a method for using a cDNA to detect the differential expression of a nucleic acid in a sample comprising hybridizing a probe to the nucleic acids, thereby forming hybridization complexes and comparing hybridization complex formation with a standard, wherein the comparison indicates the differential expression of the cDNA in the sample. In one aspect, the method of detection further comprises amplifying the nucleic acids of the sample prior to hybridization. In another aspect, the method showing differential expression of the cDNA is used to diagnose a cancer or complications thereof.

The invention provides a method for using a cDNA to screen a library or plurality of molecules or compounds to identify at least one ligand which specifically binds the cDNA, the method comprising combining the cDNA with the molecules or compounds under conditions to allow specific binding and detecting specific binding to the cDNA, thereby identifying a ligand which specifically binds the cDNA. In one aspect, the molecules or compounds are selected from artificial chromosome constructions, antisense molecules, DNA molecules, peptides, peptide nucleic acids, proteins, regulatory molecules, RNA molecules, repressors, and transcription factors.

The invention provides a method for using a cDNA to purify a ligand which specifically binds the cDNA, the method comprising attaching the cDNA to a substrate, contacting the cDNA with a sample under conditions to allow specific binding, and dissociating the ligand from the cDNA, thereby obtaining purified ligand.

The invention provides a purified protein comprising a polypeptide having an amino acid sequence of SEQ ID NO:1. It also provides variants encoded by polynucleotides having at least 84% identity to the polypeptide having the amino acid sequence of SEQ ID NO:1, antigenic determinants of the protein selected from about L280 to about K295 and from about A17 to about W32, biologically active portions of the protein selected from about L114 to about S333; from about Q132 to about A143, from about L275 to about G285, and from about Y296 to about K314 of SEQ ID NO:1, and array elements consisting of SEQ ID NO:1.

The invention further provides a composition comprising the purified protein and a labeling moiety or a pharmaceutical carrier and a substrate upon which the protein has been immobilized. A method for diagnosing cancer comprising performing an assay to quantify the amount of the protein of claim 1 expressed in a sample and comparing the amount of protein expressed to standards, thereby diagnosing a disorder of cell proliferation, in particular lung cancer. The invention provides a method for using a protein to screen a library or a plurality of molecules or compounds to identify at least one ligand, the method comprising combining the protein with the molecules or compounds under conditions to allow specific binding and detecting specific binding, thereby identifying a ligand which specifically binds the protein. In one aspect, the molecules or compounds are selected from agonists, antagonists, antibodies, DNA molecules, small drug molecules, immunoglobulins, inhibitors, mimetics, peptides, peptide nucleic acids, proteins, and RNA molecules. In another aspect, the ligand is used to treat a subject with a cancer or complications thereof. The invention also provides an antagonist which specifically binds the protein having the amino acid sequence of SEQ ID NO:1. The invention further provides a small drug molecule which specifically binds the protein having the amino acid sequence of SEQ ID NO:1.

The invention provides a method for using a protein to screen a plurality of antibodies to identify an antibody which specifically binds the protein comprising contacting a plurality of antibodies with the protein under conditions to form an antibody:protein complex, and dissociating the antibody from the antibody:protein complex, thereby obtaining antibody which specifically binds the protein.

The invention also provides methods for using a protein to prepare and purify polyclonal and monoclonal antibodies which specifically bind the protein. The method for preparing a polyclonal antibody comprises immunizing a animal with protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified polyclonal antibodies. The method for preparing a monoclonal antibodies comprises immunizing a animal with a protein under conditions to elicit an antibody response, isolating antibody producing cells from the animal, fusing the antibody producing cells with immortalized cells in culture to form monoclonal antibody producing hybridoma cells, culturing the hybridoma cells, and isolating monoclonal antibodies from culture.

The invention provides purified antibodies which bind specifically to a protein. The invention also provides a method for using an antibody to detect expression of a protein in a sample, the method comprising combining the antibody with a sample under conditions for formation of antibody:protein complexes, and detecting complex formation, wherein complex formation indicates expression of the protein in the sample. In one aspect, the amount of complex formation when compared to standards is diagnostic of a disorder of cell proliferation, in particular lung cancer.

The invention provides a method for immunopurification of a protein comprising attaching an antibody to a substrate, exposing the antibody to a sample containing protein under conditions to allow antibody:protein complexes to form, dissociating the protein from the complex, and collecting purified protein. The invention also provides an array upon which a cDNA encoding LCAP, LCAP, or an antibody which specifically binds LCAP are immobilized.

The invention provides a method for inserting a heterologous marker gene into the genomic DNA of a mammal to disrupt the expression of the endogenous polynucleotide. The invention also provides a method for using a cDNA to produce a mammalian model system, the method comprising constructing a vector containing the cDNA selected from SEQ ID NOs:2-17, transforming the vector into an embryonic stem cell, selecting a transformed embryonic stem cell, microinjecting the transformed embryonic stem cell into a mammalian blastocyst, thereby forming a chimeric blastocyst, transferring the chimeric blastocyst into a pseudopregnant dam, wherein the dam gives birth to a chimeric offspring containing the cDNA in its germ line, and breeding the chimeric mammal to produce a homozygous, mammalian model system.

### BRIEF DESCRIPTION OF THE FIGURES AND TABLES

FIGS. 1A, 1B, 1C and 1D show the amino acid sequence (SEQ ID NO:1) and nucleic acid sequence (SEQ ID NO:2) of a new human cathepsin. The alignment was produced using MACDNASIS PRO software (Hitachi Software Engineering, South San Francisco Calif.).

FIGS. 2A and 2B show the amino acid sequence alignments among LCAP (SEQ ID NO:1), human cathepsin L (GI 29715; SEQ ID NO:18) and porcine cathepsin L (GI 1468964; SEQ ID NO:19) produced using the MEGALIGN program of LASERGENE software (DNASTAR, Madison Wis.).

FIG. 3 shows expression of LCAP as determined using quantitative real-time polymerase chain reaction (QPCR; Applied Biosystems (ABI), Foster City Calif.), the oligonucleotide extending from about nucleotide 1011 to about nucleotide 1086 of SEQ ID NO:2, and biopsied, matched normal/tumor lung tissues obtained from the Roy Castle International Centre for Lung Cancer Research (RCIC), Liverpool UK.

FIG. 4 shows expression of LCAP as determined using QPCR (ABI), the oligonucleotide extending from about nucleotide 1011 to about nucleotide 1086 of SEQ ID NO:2, and normal human tissues (Clontech Laboratories, Palo Alto Calif.).

### DESCRIPTION OF THE INVENTION

It is understood that this invention is not limited to the particular machines, materials and methods described. It is also to be understood that the terminology used herein is for the purpose of describing particular embodiments and is not intended to limit the scope of the present invention which will be limited only by the appended claims. As used herein, the singular forms “a”, “an”, and “the” include plural reference unless the context clearly dictates otherwise. For example, a reference to “a host cell” includes a plurality of such host cells known to those skilled in the art.

Unless defined otherwise, all technical and scientific terms used herein have the same meanings as commonly understood by one of ordinary skill in the art to which this invention belongs. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“Antibody” refers to intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment, and an antibody-peptide fusion protein.

“Antigenic determinant” refers to an antigenic or immunogenic epitope, structural feature, or region of an oligopeptide, peptide, or protein which is capable of inducing formation of an antibody which specifically binds the protein. Biological activity is not a prerequisite for immunogenicity.

“Array” refers to an ordered arrangement of at least two cDNAs, proteins, or antibodies on a substrate. At least one of the cDNAs, proteins, or antibodies represents a control or standard, and the other cDNA, protein, or antibody is of diagnostic or therapeutic interest. The arrangement of at least two and up to about 40,000 cDNAs, proteins, or antibodies on the substrate assures that the size and signal intensity of each labeled complex, formed between each cDNA and at least one nucleic acid, each protein and at least one ligand or antibody, or each antibody and at least one protein to which the antibody specifically binds, is individually distinguishable.

The “complement” of a cDNA of the Sequence Listing refers to a nucleic acid molecule which is completely complementary over its full length and which will hybridize to a nucleic acid molecule under conditions of high stringency.

“cDNA” refers to an isolated polynucleotide, nucleic acid molecule, or any fragment thereof that contains from about 400 to about 12,000 nucleotides. It may have originated recombinantly or synthetically, may be double-stranded or single-stranded, may represent coding and noncoding 3′ or 5′ sequence, and generally lacks introns.

The phrase “cDNA encoding a protein” refers to a nucleic acid whose sequence closely aligns with sequences that encode conserved regions, motifs or domains identified by employing analyses well known in the art. These analyses include BLAST (Basic Local Alignment Search Tool; Altschul (1993) J Mol Evol 36:290-300; Altschul et al. (1990) J Mol Biol 215:403-410) and BLAST2 (Altschul et al. (1997) Nucleic Acids Res 25:3389-3402) which provide identity within the conserved region. Brenner et al. (1998; Proc Natl Acad Sci 95:6073-6078) who analyzed BLAST for its ability to identify structural homologs by sequence identity found 30% identity is a reliable threshold for sequence alignments of at least 150 residues and 40% is a reasonable threshold for alignments of at least 70 residues (Brenner, page 6076, column 2).

A “composition” refers to the polynucleotide and a labeling moiety; a purified protein and a pharmaceutical carrier or a heterologous, labeling or purification moiety; an antibody and a labeling moiety or pharmaceutical agent; and the like.

“Derivative” refers to a cDNA or a protein that has been subjected to a chemical modification. Derivatization of a cDNA can involve substitution of a nontraditional base such as queosine or of an analog such as hypoxanthine. These substitutions are well known in the art. Derivatization of a protein involves the replacement of a hydrogen by an acetyl, acyl, alkyl, amino, formyl, or morpholino group. Derivative molecules retain the biological activities of the naturally occurring molecules but may confer advantages such as longer lifespan or enhanced activity.

“Differential expression” refers to an increased or upregulated or a decreased or downregulated expression as detected by absence, presence, or at least two-fold change in the amount of transcribed messenger RNA or translated protein in a sample.

“Disorder” refers to conditions, diseases or syndromes in which the cDNA encoding LCAP and LCAP are differentially expressed; these include disorders of cell proliferation, and in particular cancers of the bladder, breast, colon, kidney, lung, lymph node, ovary and uterus and immune complications associated with any of these cancers.

An “expression profile” is a representation of gene expression in a sample. A nucleic acid expression profile is produced using sequencing, hybridization, or amplification technologies and mRNAs or cDNAs from a sample. A protein expression profile, although time delayed, mirrors the nucleic acid expression profile and uses two-dimensional polyacrylamide electrophoresis (2D-PAGE, mass spectrophotometry (MS), enzyme-linked immunosorbent assays (ELISAs), radioimmunoassays (RIAs), and fluorescence activated cell sorting (FACS) or arrays and labeling moieties or antibodies to detect expression in a sample. The nucleic acids, proteins, or antibodies may be used in solution or attached to a substrate, and their detection is based on methods and labeling moieties well known in the art.

“Fragment” refers to a chain of consecutive nucleotides from about 50 to about 4000 base pairs in length. Fragments may be used in PCR or hybridization technologies to identify related nucleic acid molecules and in binding assays to screen for a ligand. Such ligands are useful as therapeutics to regulate replication, transcription or translation.

“LCAP” refers to a cathepsin having the an amino acid sequence that is greater than 82% homologous to SEQ ID NO:1 and was obtained from any species including bovine, ovine, murine, equine, and preferably the human species, and from any source, whether natural, synthetic, semi-synthetic, or recombinant.

A “hybridization complex” is formed between a polynucleotide of the invention and a nucleic acid of a sample when the purines of one molecule hydrogen bond with the pyrimidines of the complementary molecule, e.g., 5′-A-G-T-C-3′ base pairs with its complete complement, 3′-T-C-A-G-5′. The degree of complementarity and the use of nucleotide analogs affect the efficiency and stringency of hybridization reactions.

“Identity” as applied to sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul (1997, supra). BLAST2 may be used in a standardized and reproducible way to insert gaps in one of the sequences in order to optimize alignment and to achieve a more meaningful comparison between them. “Similarity” uses the same algorithms but takes conservative substitution of residues into account. In proteins, similarity exceeds identity in that substitution of a valine for a leucine or isoleucine, is counted in calculating the reported percentage. Substitutions which are considered to be conservative are well known in the art.

“Isolated or “purified” refers to any molecule or compound that is separated from its natural environment and is from about 60% free to about 90% free from other components with which it is naturally associated.

“Labeling moiety” refers to any reporter molecule including radionuclides, enzymes, fluorescent, chemiluminescent, or chromogenic agents, substrates, cofactors, inhibitors, or magnetic particles than can be attached to or incorporated into a polynucleotide, protein, or antibody. Visible labels include but are not limited to anthocyanins, green fluorescent protein (GFP), β glucuronidase, luciferase, Cy3 and Cy5, and the like. Radioactive markers include radioactive forms of hydrogen, iodine, phosphorous, sulfur, and the like.

“Ligand” refers to any agent, molecule, or compound which will bind specifically to a polynucleotide or to an epitope of a protein. Such ligands stabilize or modulate the activity of polynucleotides or proteins and may be composed of inorganic and/or organic substances including minerals, cofactors, nucleic acids, proteins, carbohydrates, fats, and lipids.

“Oligonucleotide” refers a single-stranded molecule from about 18 to about 60 nucleotides in length which includes both 5′ and 3′ primers and the intervening nucleotide sequence which is used as the probe in hybridization or amplification technologies or in regulation of replication, transcription or translation. Equivalent terms are amplicon, amplimer, primer, and oligomer.

“Post-translational modification” of a protein can involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like.

“Probe” refers to a cDNA that hybridizes to at least one nucleic acid in a sample. Where targets are single-stranded, probes are complementary single strands. Probes can be labeled with reporter molecules for use in hybridization reactions including Southern, northern, in situ, dot blot, array, and like technologies or in screening assays.

“Protein” refers to a polypeptide or any portion thereof. A “portion” of a protein refers to that length of amino acid sequence which would retain at least one biological activity, a domain identified by PFAM or PRINTS analysis or an antigenic determinant of the protein identified using Kyte-Doolittle algorithms of the PROTEAN program (DNASTAR, Madison Wis.). An “oligopeptide” is an amino acid sequence from about five residues to about 15 residues that is used as part of a fusion protein to produce an antibody.

“Sample” is used in its broadest sense as containing nucleic acids, proteins, and antibodies. A sample may comprise a bodily fluid such as ascites, blood, lymph, saliva, semen, spinal, sputum, tears, urine and the like; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue, a tissue biopsy, or a tissue print; buccal cells, skin, hair, a hair follicle; and the like.

“Specific binding” refers to a special and precise interaction between two molecules which is dependent upon their structure, particularly their molecular side groups. For example, the intercalation of a regulatory protein into the major groove of a DNA molecule or the binding between an epitope of a protein and an agonist, antagonist, or antibody.

“Substrate” refers to any rigid or semi-rigid support to which cDNAs, proteins, or antibodies are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

A “transcript image” (TI) is a profile of gene transcription activity in a particular tissue at a particular time. TI provides assessment of the relative abundance of expressed polynucleotides in the cDNA libraries of an EST database as described in U.S. Pat. No. 5,840,484, incorporated herein by reference.

“Variant” refers to molecules that are recognized variations of a protein or the polynucleotides that encode it. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid or its secondary, tertiary, or quaternary structure.

### THE INVENTION

The invention is human cathepsin (LCAP), its encoding cDNA, and an antibody which specifically binds the protease and their use in the characterization, diagnosis, prognosis, treatment and evaluation of treatment of disorders of cell proliferation, particularly lung cancer and complications of lung cancer. U.S. Pat. No. 6,033,893 is incorporated in its entirety by reference herein.

The cDNA encoding LCAP of the present invention was first identified in Incyte clone 347021 from a thymus cDNA library (THYMNOT02) using a computer search for amino acid sequence alignments. The cDNA comprising the nucleic acid sequence of SEQ ID NO:2 encompasses the following overlapping and/or extended cDNAs: Incyte Clones 347021H1, 347021R1, 389479H1, and 389479T6 from THYMNOT02 and 2554720H1, 2554720CA2 and 2555589F6 from THYMNOT03 which are SEQ ID NOs:3-9, respectively. The clones 2554720H1, 2554720CA2 and 2555589F6 (SEQ ID NOs:7-9) have been resequenced at least once, and each has been designated a verified reagent. The cDNA encoding LCAP maps to chromosome 9q21-22. One useful oligonucleotide of SEQ ID NO:2 extends from about nucleotide 1011 to about nucleotide 1086.

Electronic northern analysis showed expression of transcripts corresponding to nucleic acid sequence SEQ ID NO:2 in 211 cDNA libraries, 75% of which were tissues associated with cell proliferation; in particular, 37% with cancer of the bladder, breast, colon, kidney, lung, lymph node, ovary and uterus; 25%, with immune response; and 13%, with fetal or infant development. Transcript imaging as demonstrated in

EXAMPLE V shows the expression of transcripts corresponding to SEQ ID NO:2 in libraries made from tissues of patients with squamous cell carcinoma. When microarray analysis showed the differential expression of SEQ ID NO:2 in 8 of 26 non-small cell lung tumors (data not shown), QPCR was performed.

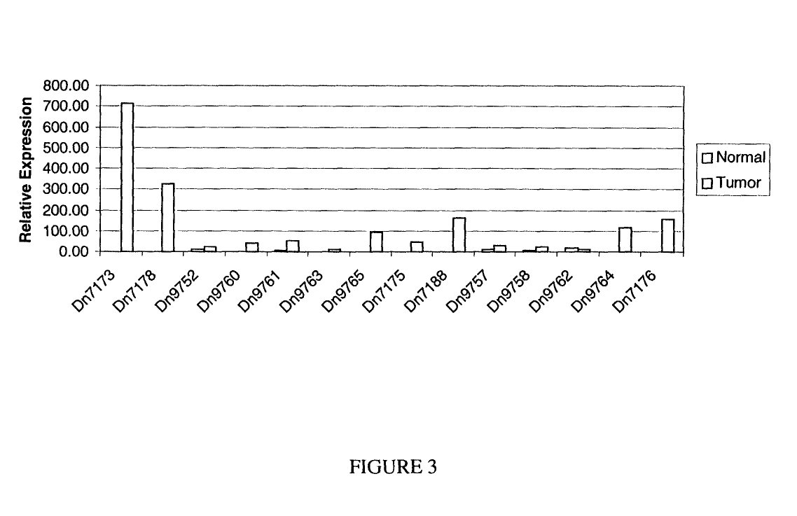

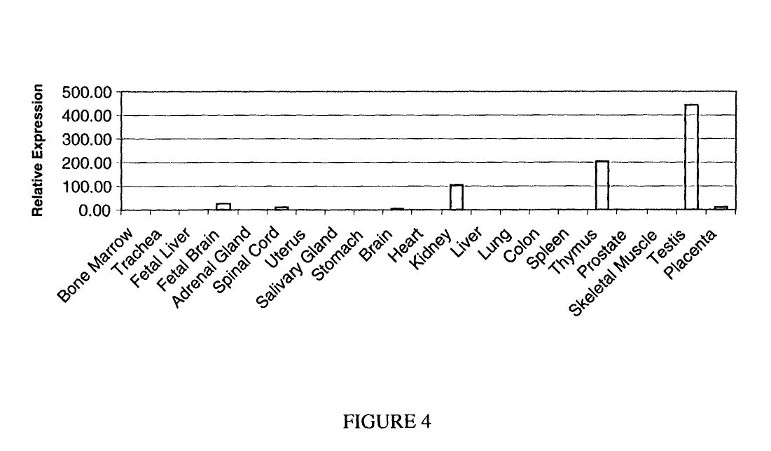

FIGS. 3 and 4 show QPCR results examining the expression of LCAP across a panel of matched lung tumor and cytologically normal tissues, and a panel of non-lung normal tissues, respectively. FIG. 3 shows expression of LCAP as determined using QPCR (Applied Biosystems (ABI), Foster City Calif.), the oligonucleotide extending from about nucleotide 1011 to nucleotide 1086 of SEQ ID NO:2, and biopsied, matched normal/tumor lung tissues obtained from the Roy Castle International Centre for Lung Cancer Research (RCIC), Liverpool UK. All results were standardized to the cytologically normal lung tissue from patient donor 7178. SEQ ID NO:2 was differentially expressed in 13 of 14 non-small lung tumor tissues as compared with expression in 14 matched, cytologically normal lung tissues from the same patient donors. Donor tissues are described in EXAMPLE VII.

FIG. 4 shows expression of LCAP as determined using QPCR (ABI), the oligonucleotide extending from about nucleotide 1011 to nucleotide 1086 of SEQ ID NO:2, and normal tissues obtained from Clontech Laboratories. All results were standardized to the normal trachea. As shown in FIG. 4, SEQ ID NO:2 was expressed in fetal brain and placenta and in normal spinal cord, brain, kidney, thymus and testis. The lung and non-lung tissues are described in EXAMPLE VII under the section entitled Tissue Sample Preparation.

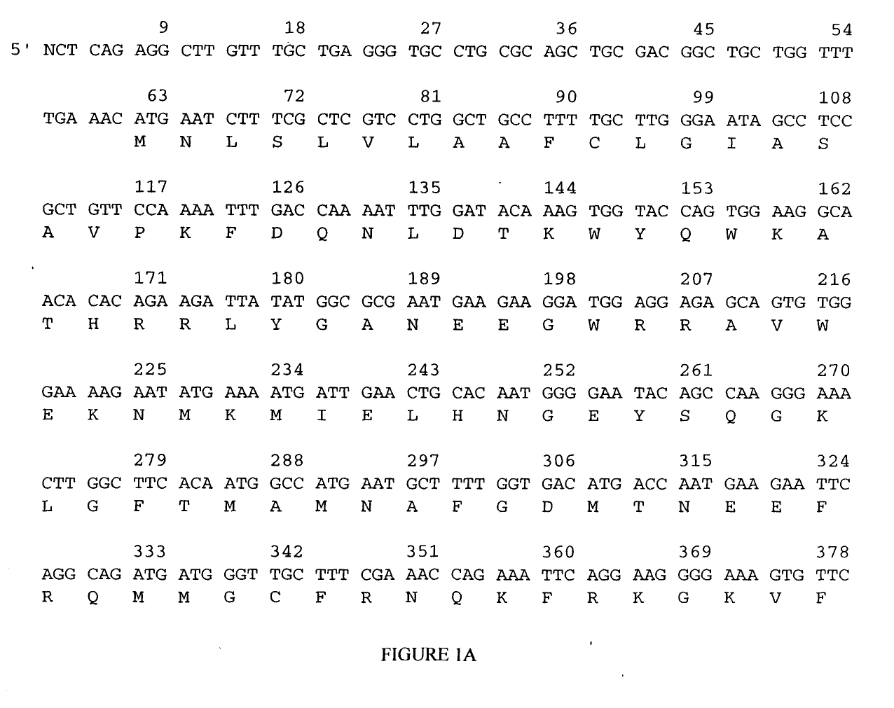

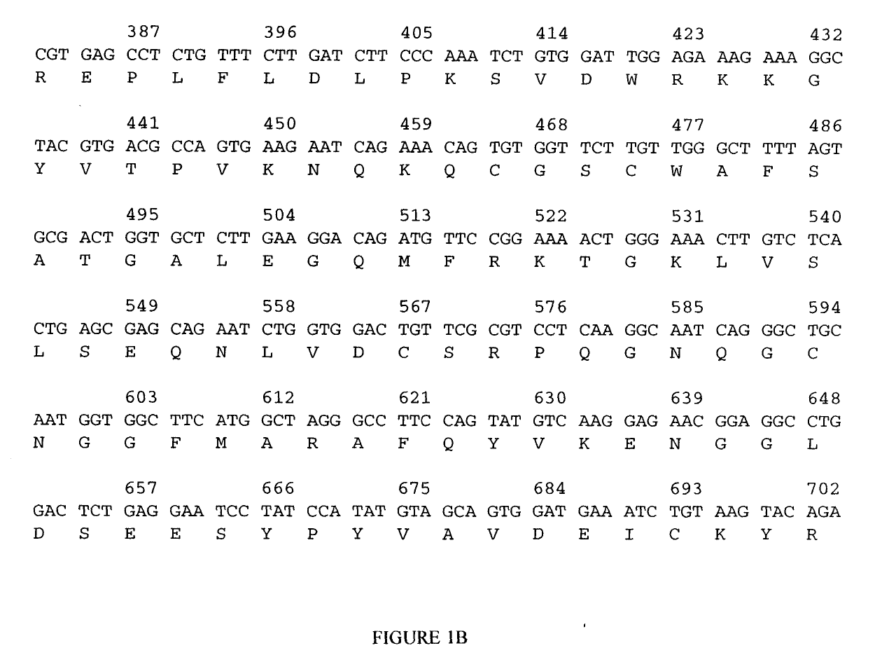

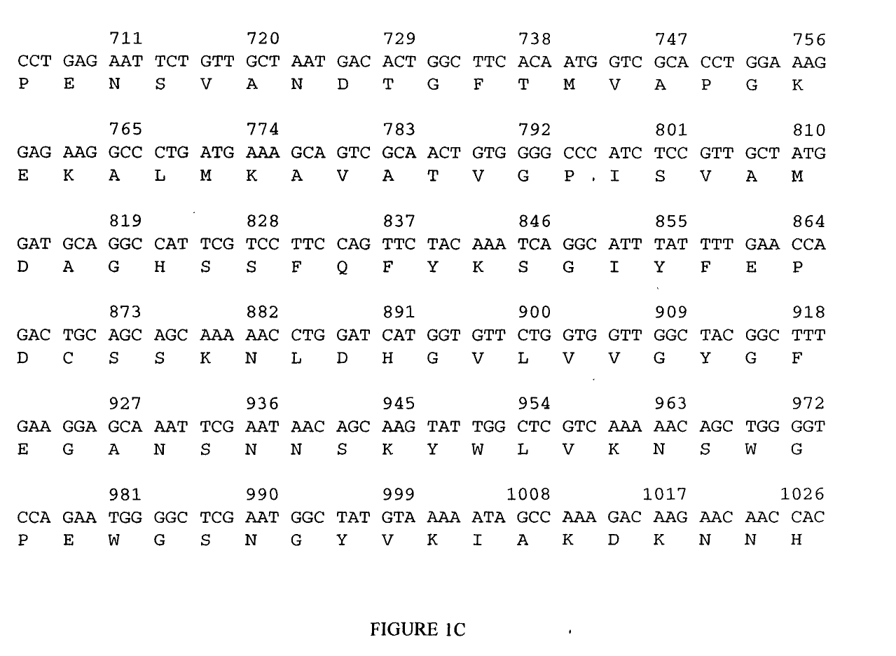

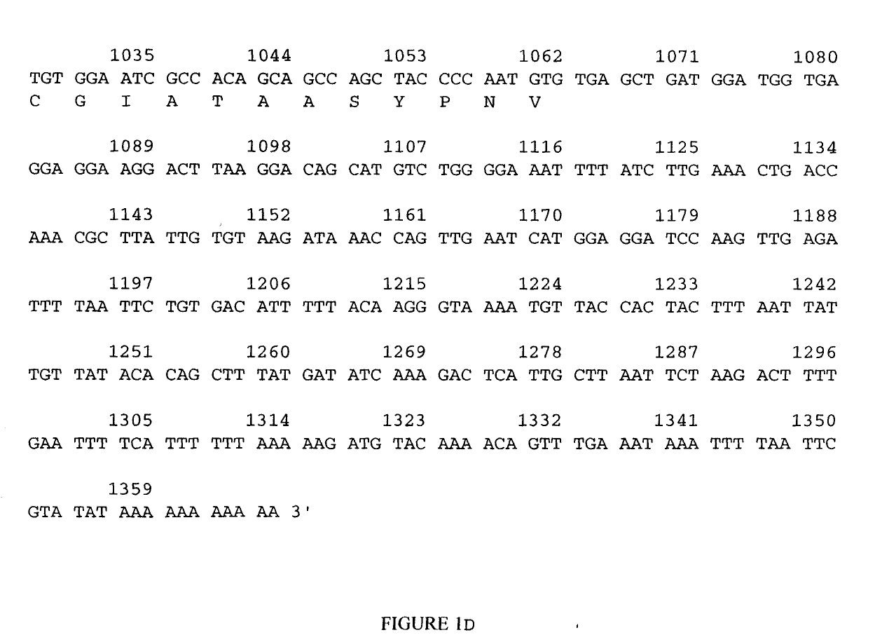

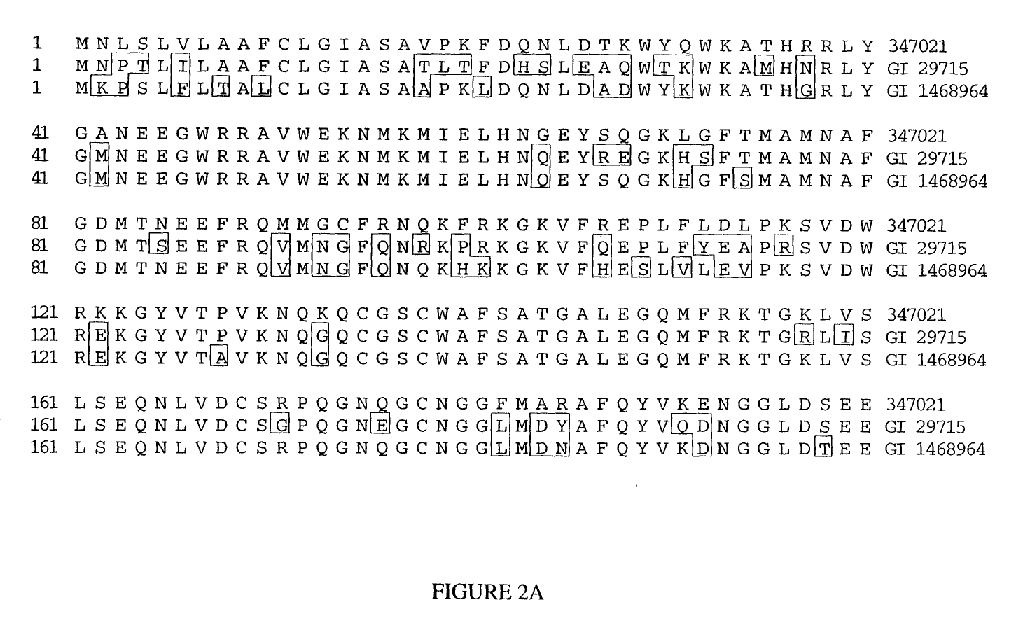

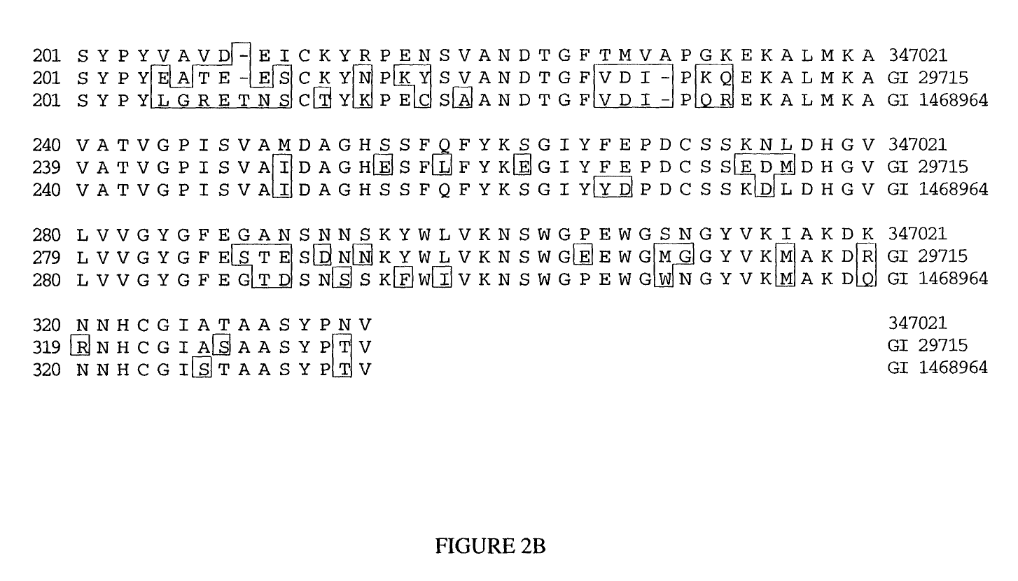

In one embodiment, the invention encompasses a protein comprising the amino acid sequence of SEQ ID NO:1, as shown in FIGS. 1A-1D. As shown in the figures or sequence listing, LCAP is 334 amino acids in length and is the preproform of the enzyme. LCAP has a signal sequence from residue 1 to residue 17; three potential N glycosylation sites at N2, N221 and N292; five potential phosphorylation sites at T35, T84, T155, S160, and S271; a cysteine protease domain from L114 to S333; and three potential thiol protease active sites, specifically Q₁₃₂KQCGSCWAFSA, L₂₇₅DHGVLVVGYG, and Y₂₉₆WLVKNSWGPEWGSNGYVK. The specific catalytic residue for each of the thiol protease active sites is underlined. As shown in FIGS. 2A-2B, LCAP has chemical and structural homology with human and pig cathepsins, GI 29715 (SEQ ID NO:18) and GI1468964 (SEQ ID NO:19), respectively. In particular, LCAP shares 77% identity with human cathepsin, and 81% identity with pig cathepsin. Useful biologically active portions of LCAP extend from about L114 to about S333; from about Q132 to about A143, from about L275 to about G285, and from about Y296 to about K314 of SEQ ID NO:1. Useful antigenic determinant which have been used to make an antibodies extend from about L280 to about K295 and from about A17 to about W32.

Mammalian variants of the cDNA encoding LCAP were identified using BLAST2 with default parameters and the ZOOSEQ databases (Incyte Genomics, Palo Alto Calif.). These non-human, homologous cDNAs have about identity to all or part of the coding region of the human cDNA as shown in the table below. As shown in FIG. 1, the coding region for the human cDNA extends from nucleotide 61 to nucleotide 1062 and the first 17 nucleotides of the coding sequence are signal sequence. Therefore, the mature cathepsin coding region is encompassed by the cDNAs of rat, dog, and mouse (SEQ ID NOs:10, 13, and 16, respectively). The first column represents the SEQ ID NO: for variant cDNAs; the second column, the Incyte ID for the variant cDNAs; the third column, the species; the fourth column, the percent identity to the human cDNA; and the fifth column, the nucleotide alignment of the variant cDNA to the human cDNA.

| SEQ ID_{Var} | Incyte ID_{Var} | Species | Identity | Nt_{H} Alignment |
|---|---|---|---|---|
| 10 | 223055_Rn.10 | Rat | 80% | 76-1234 |
| 11 | 703912760 | Rat | 80% | 76-677 |
| 12 | 003170_Mf.1 | Monkey | 84% | 639-1092 |
| 13 | 007499_Cf.1 | Dog | 86% | 51-1327 |
| 14 | 704138617 | Dog | 87% | 51-816 |
| 15 | 703985027J1 | Dog | 86% | 510-1318 |
| 16 | 085657_Mm.1 | Mouse | 80% | 52-1286 |
| 17 | 701326170 | Mouse | 87% | 755-1327 |

It will be appreciated by those skilled in the art that as a result of the degeneracy of the genetic code, a multitude of cDNAs encoding LCAP, some bearing minimal similarity to the cDNAs of any known and naturally occurring gene, may be produced. Thus, the invention contemplates each and every possible variation of cDNA that could be made by selecting combinations based on possible codon choices. These combinations are made in accordance with the standard triplet genetic code as applied to the polynucleotide encoding naturally occurring LCAP, and all such variations are to be considered as being specifically disclosed.

The cDNAs of SEQ ID NOs:2-17 may be used in hybridization, amplification, and screening technologies to identify and distinguish among SEQ ID NO:2 and related molecules in a sample. The mammalian cDNAs, SEQ ID NOs:10-17, may be used to produce transgenic cell lines or organisms which are model systems for human lung cancer and upon which the toxicity and efficacy of potential therapeutic treatments may be tested. Toxicology studies, clinical trials, and subject/patient treatment profiles may be performed and monitored using the cDNAs, proteins, antibodies and molecules and compounds identified using the cDNAs and proteins of the present invention.

Characterization and Use of the Invention

cDNA Libraries

In a particular embodiment disclosed herein, mRNA is isolated from mammalian cells and tissues using methods which are well known to those skilled in the art and used to prepare the cDNA libraries. The Incyte cDNAs were isolated from mammalian cDNA libraries prepared as described in the EXAMPLES. The consensus sequence is present in a single clone insert, or chemically assembled, based on the electronic assembly from sequenced fragments including Incyte cDNAs and extension and/or shotgun sequences. Computer programs, such as PHRAP (P Green, University of Washington, Seattle Wash.) and the AUTOASSEMBLER application (ABI), are used in sequence assembly and are described in EXAMPLE V. After verification of the 5′ and 3′ sequence, at least one representative cDNA which encodes LCAP is designated a reagent for research and development.

Sequencing

Methods for sequencing nucleic acids are well known in the art and may be used to practice any of the embodiments of the invention. These methods employ enzymes such as the Klenow fragment of DNA polymerase I, SEQUENASE, Taq DNA polymerase and thermostable T7 DNA polymerase (Amersham Biosciences (APB), Piscataway N.J.), or combinations of commercially available polymerases and proofreading exonucleases (Invitrogen, San Diego Calif.). Sequence preparation is automated with machines such as the MICROLAB 2200 system (Hamilton, Reno Nev.) and the DNA ENGINE thermal cycler (MJ Research, Watertown Mass.) and sequencing, with the PRISM 3700, 377 or 373 DNA sequencing systems (ABI) or the MEGABACE 1000 DNA sequencing system (APB).

The nucleic acid sequences of the cDNAs presented in the Sequence Listing were prepared by such automated methods and may contain occasional sequencing errors and unidentified nucleotides (N) that reflect state-of-the-art technology at the time the cDNA was sequenced. Occasional sequencing errors and Ns may be resolved and SNPs verified either by resequencing the cDNA or using algorithms to compare multiple sequences; both of these techniques are well known to those skilled in the art who wish to practice the invention. The sequences may be analyzed using a variety of algorithms described in Ausubel et al. (1997; Short Protocols in Molecular Biology, John Wiley & Sons, New York N.Y., unit 7.7) and in Meyers (1995; Molecular Biology and Biotechnology, Wiley VCH, New York N.Y., pp. 856-853).

Shotgun sequencing may also be used to complete the sequence of a particular cloned insert of interest. Shotgun strategy involves randomly breaking the original insert into segments of various sizes and cloning these fragments into vectors. The fragments are sequenced and reassembled using overlapping ends until the entire sequence of the original insert is known. Shotgun sequencing methods are well known in the art and use thermostable DNA polymerases, heat-labile DNA polymerases, and primers chosen from representative regions flanking the cDNAs of interest. Incomplete assembled sequences are inspected for identity using various algorithms or programs such as CONSED (Gordon (1998) Genome Res 8:195-202) which are well known in the art. Contaminating sequences, including vector or chimeric sequences, can be removed, and deleted sequences can be restored to complete the assembled, finished sequences.

Extension of a Nucleic Acid Sequence

The sequences of the invention may be extended using various PCR-based methods known in the art. For example, the GENEAMP XL PCR kit (ABI), nested primers, and commercially available cDNA or genomic DNA libraries may be used to extend the nucleic acid sequence. For all PCR-based methods, primers may be designed using commercially available software, such as OLIGO primer analysis software (Molecular Biology Insights, Cascade Colo.) to be about 22 to 30 nucleotides in length, to have a GC content of −15 about 50% or more, and to anneal to a target molecule at temperatures from about 55C to about 68C. When extending a sequence to recover regulatory elements, it is preferable to use genomic, rather than cDNA libraries.

Hybridization

The cDNA and fragments thereof can be used in hybridization technologies for various purposes. A probe may be designed or derived from unique regions such as the 5′ regulatory region or from a nonconserved region (i.e., 5′ or 3′ of the nucleotides encoding the conserved catalytic domain of the protein) and used in protocols to identify naturally occurring molecules encoding the LCAP, allelic variants, or related molecules. The probe may be DNA or RNA, may be single-stranded, and should have at least 50% sequence identity to any of the nucleic acid sequences, SEQ ID NOs:2-17. Hybridization probes may be produced using oligolabeling, nick-translation, end-labeling, or PCR amplification in the presence of a reporter molecule. A vector containing the cDNA or a fragment thereof may be used to produce an mRNA probe in vitro by addition of an RNA polymerase and labeled nucleotides. These procedures may be conducted using commercially available kits such as those provided by APB.

The stringency of hybridization is determined by G+C content of the probe, salt concentration, and temperature. In particular, stringency can be increased by reducing the concentration of salt or raising the hybridization temperature. Hybridization can be performed at low stringency with buffers, such as 5×SSC with 1% sodium dodecyl sulfate (SDS) at 60C, which permits the formation of a hybridization complex between nucleic acid sequences that contain some mismatches. Subsequent washes are performed at higher stringency with buffers such as 0.2×SSC with 0.1% SDS at either 45C (medium stringency) or 68C (high stringency). At high stringency, hybridization complexes will remain stable only where the nucleic acids are completely complementary. In some membrane-based hybridizations, from about 35% to about 50% formamide can be added to the hybridization solution to reduce the temperature at which hybridization is performed. Background signals can be reduced by the use of detergents such as Sarkosyl or TRITON X-100 (Sigma-Aldrich, St. Louis Mo.) and a blocking agent such as denatured salmon sperm DNA. Selection of components and conditions for hybridization are well known to those skilled in the art and are reviewed in Ausubel (supra) and Sambrook et al. (1989) Molecular Cloning, A Laboratory Manual, Cold Spring Harbor Press, Plainview N.Y.

Arrays may be prepared and analyzed using methods well known in the art. Oligonucleotides or cDNAs may be used as hybridization probes or targets to monitor the expression level of large numbers of genes simultaneously or to identify genetic variants, mutations, and single nucleotide polymorphisms. Arrays may be used to determine gene function; to understand the genetic basis of a condition, disease, or disorder; to diagnose a condition, disease, or disorder; and to develop and monitor the activities of therapeutic agents. (See, e.g., U.S. Pat. No. 5,474,796; Schena et al. (1996) Proc Natl Acad Sci 93:10614-10619; Heller et al. (1997) Proc Natl Acad Sci 94:2150-2155; U.S. Pat. No. 5,605,662.)

Hybridization probes are also useful in mapping the naturally occurring genomic sequence. The probes may be hybridized to a particular chromosome, a specific region of a chromosome, or an artificial chromosome construction. Such constructions include human artificial chromosomes, yeast artificial chromosomes, bacterial artificial chromosomes, bacterial P1 constructions, or the cDNAs of libraries made from single chromosomes.

QPCR

QPCR is a method for quantifying a nucleic acid molecule based on detection of a fluorescent signal produced during PCR amplification (Gibson et al. (1996) Genome Res 6:995-1001; Heid et al. (1996) Genome Res 6:986-994). Amplification is carried out on machines such as the PRISM 7700 detection system (ABI) which consists of a 96-well thermal cycler connected to a laser and charge-coupled device (CCD) optics system. To perform QPCR, a PCR reaction is carried out in the presence of a doubly labeled probe. The probe, which is designed to anneal between the standard forward and reverse PCR primers, is labeled at the 5′ end by a flourogenic reporter dye such as 6-carboxyfluorescein (6-FAM) and at the 3′ end by a quencher molecule such as 6-carboxy-tetramethyl-rhodamine (TAMRA). As long as the probe is intact, the 3′ quencher extinguishes fluorescence by the 5′ reporter. However, during each primer extension cycle, the annealed probe is degraded as a result of the intrinsic 5′ to 3′ nuclease activity of Taq polymerase (Holland et al. (1991) Proc Natl Acad Sci 88:7276-7280). This degradation separates the reporter from the quencher, and fluorescence is detected every few seconds by the CCD. The higher the starting copy number of the nucleic acid, the sooner a significant increase in fluorescence is observed. A cycle threshold (C_{T}) value, representing the cycle number at which the PCR product crosses a fixed threshold of detection is determined by the instrument software. The C_{T} is inversely proportional to the copy number of the template and can therefore be used to calculate either the relative or absolute initial concentration of the nucleic acid molecule in the sample. The relative concentration of two different molecules can be calculated by determining their respective C_{T} values (comparative C_{T} method). Alternatively, the absolute concentration of the nucleic acid molecule can be calculated by constructing a standard curve using a housekeeping molecule of known concentration. The process of calculating C_{T}s, preparing a standard curve, and determining starting copy number is performed using SEQUENCE DETECTOR 1.7 software (ABI).

Expression

Any one of a multitude of cDNAs encoding LCAP may be cloned into a vector and used to express the protein, or portions thereof, in host cells. The nucleic acid sequence can be engineered by such methods as DNA shuffling (U.S. Pat. No. 5,830,721) and site-directed mutagenesis to create new restriction sites, alter glycosylation patterns, change codon preference to increase expression in a particular host, produce splice variants, extend half-life, and the like. The expression vector may contain transcriptional and translational control elements (promoters, enhancers, specific initiation signals, and polyadenylated 3′ sequence) from various sources which have been selected for their efficiency in a particular host. The vector, cDNA, and regulatory elements are combined using in vitro recombinant DNA techniques, synthetic techniques, and/or in vivo genetic recombination techniques well known in the art and described in Sambrook (supra, ch. 4, 8, 16 and 17).

A variety of host systems may be transformed with an expression vector. These include, but are not limited to, bacteria transformed with recombinant bacteriophage, plasmid, or cosmid DNA expression vectors; yeast transformed with yeast expression vectors; insect cell systems transformed with baculovirus expression vectors or plant cell systems transformed with expression vectors containing viral and/or bacterial elements (Ausubel supra, unit 16). In mammalian cell systems, an adenovirus transcription/translation complex may be utilized. After sequences are ligated into the E1 or E3 region of the viral genome, the infective virus is used to transform and express the protein in host cells. The Rous sarcoma virus enhancer or SV40 or EBV-based vectors may also be used for high-level protein expression.

Routine cloning, subcloning, and propagation of nucleic acid sequences can be achieved using the multifunctional pBLUESCRIPT vector (Stratagene, La Jolla Calif.) or pSPORT1 plasmid (Invitrogen). Introduction of a nucleic acid sequence into the multiple cloning site of these vectors disrupts the lacZ gene and allows colorimetric screening for transformed bacteria. In addition, these vectors may be useful for in vitro transcription, dideoxy sequencing, single strand rescue with helper phage, and creation of nested deletions in the cloned sequence.

For long term production of recombinant proteins, the vector can be stably transformed into cell lines along with a selectable or visible marker gene on the same or on a separate vector. After transformation, cells are allowed to grow for about 1 to 2 days in enriched media and then are transferred to selective media. Selectable markers, antimetabolite, antibiotic, or herbicide resistance genes, confer resistance to the relevant selective agent and allow growth and recovery of cells which successfully express the introduced sequences. Resistant clones identified either by survival on selective media or by the expression of visible markers may be propagated using culture techniques. Visible markers are also used to estimate the amount of protein expressed by the introduced genes. Verification that the host cell contains the desired cDNA is based on DNA-DNA or DNA-RNA hybridizations or PCR amplification.

The host cell may be chosen for its ability to modify a recombinant protein in a desired fashion. Such modifications include acetylation, carboxylation, glycosylation, phosphorylation, lipidation, acylation and the like. Post-translational processing which cleaves a “prepro” form may also be used to specify protein targeting, folding, and/or activity. Different host cells available from the ATCC (Manassas Va.) which have specific cellular machinery and characteristic mechanisms for post-translational activities may be chosen to ensure the correct modification and processing of the recombinant protein.

Recovery of Proteins from Cell Culture

Heterologous moieties engineered into a vector for ease of purification include glutathione S-transferase (GST), 6xHis, FLAG, MYC, and the like. GST and 6-His are purified using commercially available affinity matrices such as immobilized glutathione and metal-chelate resins, respectively. FLAG and MYC are purified using commercially available monoclonal and polyclonal antibodies. For ease of separation following purification, a sequence encoding a proteolytic cleavage site may be part of the vector located between the protein and the heterologous moiety. Methods for recombinant protein expression and purification are discussed in Ausubel (supra, unit 16) and are commercially available.

Protein Identification Several techniques have been developed which permit rapid identification of proteins from samples using high performance liquid chromatography and mass spectrometry (MS). Beginning with a sample containing proteins, the major steps involved are: 1) proteins are separated using two-dimensional gel electrophoresis (2-DE), 2) selected proteins are excised from the gel and digested with a protease to produce a set of peptides; and 3) the peptides are subjected to mass spectral analysis to derive peptide ion mass and spectral pattern information. The MS information is used to identify the protein by comparing it with information in a protein database (Shevenko et al.(1996) Proc Natl Acad Sci 93:14440-14445).

Proteins are separated by 2DE employing isoelectric focusing (IEF) in the first dimension followed by SDS-PAGE in the second dimension. For IEF, an immobilized pH gradient strip is useful to increase reproducibility and resolution of the separation. Alternative techniques may be used to improve resolution of very basic, hydrophobic, or high molecular weight proteins. The separated proteins are detected using a stain or dye such as silver stain, Coomassie blue, or spyro red (Molecular Probes, Eugene Oreg.) that is compatible with MS. Gels may be blotted onto a PVDF membrane for western analysis and optically scanned using a STORM scanner (APB) to produce a computer-readable output which is analyzed by pattern recognition software such as MELANIE (GeneBio, Geneva, Switzerland). The software annotates individual spots by assigning a unique identifier and calculating their respective x,y coordinates, molecular masses, isoelectric points, and signal intensity. Individual spots of interest, such as those representing differentially expressed proteins, are excised and proteolytically digested with a site-specific protease such as trypsin or chymotrypsin, singly or in combination, to generate a set of small peptides, preferably in the range of 1-2 kDa. Prior to digestion, samples may be treated with reducing and alkylating agents, and following digestion, the peptides are then separated by liquid chromatography or capillary electrophoresis and analyzed using MS.

MS converts components of a sample into gaseous ions, separates the ions based on their mass-to-charge ratio, and determines relative abundance. For peptide mass fingerprinting analysis, a MALDI-TOF (Matrix Assisted Laser Desorption/Ionization-Time of Flight), ESI (Electrospray Ionization), and TOF-TOF (Time of Flight/Time of Flight) machines are used to determine a set of accurate peptide masses. Using analytical programs, such as TURBOSEQUEST software (Finnigan, San Jose Calif.), the MS data is compared against a database of theoretical MS data derived from known or predicted proteins. A minimum match of three peptide masses is usually required for reliable protein identification. If additional information is needed for identification, Tandem-MS may be used to derive information about individual peptides. In tandem-MS, a first stage of MS is performed to determine individual peptide masses. Then selected peptide ions are subjected to fragmentation using a technique such as collision induced dissociation to produce an ion series. The resulting fragmentation ions are analyzed in a second round of MS, and their spectral pattern may be used to determine a short stretch of amino acid sequence (Dancik et al. (1999) J Comput Biol 6:327-342).

Assuming the protein is represented in the database, a combination of peptide mass and fragmentation data, together with the calculated MW and pI of the protein, will usually yield an unambiguous identification. If no match is found, protein sequence can be obtained using direct chemical sequencing procedures well known in the art (cf. Creighton (1984) Proteins Structures and Molecular Properties, W H Freeman, New York N.Y.).

Chemical Synthesis of Peptides

Proteins or portions thereof may be produced not only by recombinant methods, but also by using chemical methods well known in the art. Solid phase peptide synthesis may be carried out in a batchwise or continuous flow process which sequentially adds α-amino- and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-α-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential deprotection, coupling of derivitized amino acids, and washing with dichloromethane and/or N, N-dimethylformamide. The peptide is cleaved between the peptide carboxy terminus and the linker group to yield a peptide acid or amide. (Novabiochem 1997/98 Catalog and Peptide Synthesis Handbook, San Diego Calif. pp. S1-S20). Automated synthesis may also be carried out on machines such as the 431A peptide synthesizer (ABI). A protein or portion thereof may be purified by preparative high performance liquid chromatography and its composition confirmed by amino acid analysis or by sequencing (Creighton (1984) Proteins, Structures and Molecular Properties, W H Freeman, New York N.Y.).

Antibodies

Antibodies, or immunoglobulins (Ig), are components of immune response expressed on the surface of or secreted into the circulation by B cells. The prototypical antibody is a tetramer composed of two identical heavy polypeptide chains (H-chains) and two identical light polypeptide chains (L-chains) interlinked by disulfide bonds which binds and neutralizes foreign antigens. Based on their H-chain, antibodies are classified as IgA, IgD, IgE, IgG or IgM. The most common class, IgG, is tetrameric while other classes are variants or multimers of the basic structure.

Antibodies are described in terms of their two main functional domains. Antigen recognition is mediated by the Fab (antigen binding fragment) region of the antibody, while effector functions are mediated by the Fc (crystallizable fragment) region. The binding of antibody to antigen triggers destruction of the antigen by phagocytic white blood cells such as macrophages and neutrophils. These cells express surface Fc receptors that specifically bind to the Fc region of the antibody and allow the phagocytic cells to destroy antibody-bound antigen. Fc receptors are single-pass transmembrane glycoproteins containing about 350 amino acids whose extracellular portion typically contains two or three Ig domains (Sears et al. (1990) J Immunol 144:371-378).

Preparation and Screening of Antibodies

Various hosts including mice, rats, rabbits, goats, llamas, camels, and human cell lines may be immunized by injection with an antigenic determinant. Adjuvants such as Freund's, mineral gels, and surface active substances such as lysolecithin, pluronic polyols, polyanions, peptides, oil emulsions, keyhole limpet hemacyanin (KLH; Sigma-Aldrich), and dinitrophenol may be used to increase immunological response. In humans, BCG (bacilli Calmette-Guerin) and Corynebacterium parvum are preferable. The antigenic determinant may be an oligopeptide, peptide, or protein. When the amount of antigenic determinant allows immunization to be repeated, specific polyclonal antibody with high affinity can be obtained (Klinman and Press (1975) Transplant Rev 24:41-83). Oligopepetides which may contain between about five and about fifteen amino acids identical to a portion of the endogenous protein may be fused with proteins such as KLH in order to produce antibodies to the chimeric molecule.

Monoclonal antibodies may be prepared using any technique which provides for the production of antibodies by continuous cell lines in culture. These include the hybridoma technique, the human B-cell hybridoma technique, and the EBV-hybridoma technique (Kohler et al. (1975) Nature 256:495-497; Kozbor et al. (1985) J Immunol Methods 81:3142; Cote et al. (1983) Proc Natl Acad Sci 80:2026-2030; and Cole et al. (1984) Mol Cell Biol 62:109-120).

Chimeric antibodies may be produced by techniques such as splicing of mouse antibody genes to human antibody genes to obtain a molecule with appropriate antigen specificity and biological activity (Morrison et al. (1984) Proc Natl Acad Sci 81:6851-6855; Neuberger et al. (1984) Nature 312:604-608; and Takeda et al. (1985) Nature 314:452-454). Alternatively, techniques described for antibody production may be adapted, using methods known in the art, to produce specific, single chain antibodies. Antibodies with related specificity, but of distinct idiotypic composition, may be generated by chain shuffling from random combinatorial immunoglobulin libraries (Burton (1991) Proc Natl Acad Sci 88:10134-10137). Antibody fragments which contain specific binding sites for an antigenic determinant may also be produced. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse et al. (1989) Science 246:1275-1281).

Antibodies may also be produced by inducing production in the lymphocyte population or by screening immunoglobulin libraries or panels of specific binding reagents as disclosed in Orlandi et al. (1989; Proc Natl Acad Sci 86:3833-3837) or Winter et al. (1991; Nature 349:293-299). A protein may be used in screening assays of phagemid or B-lymphocyte immunoglobulin libraries to identify antibodies having a desired specificity. Numerous protocols for competitive binding or immunoassays using either polyclonal or monoclonal antibodies with established specificities are well known in the art.

Antibody Specificity

Various methods such as Scatchard analysis combined with radioimmunoassay techniques may be used to assess the affinity of particular antibodies for a protein. Affinity is expressed as an association constant, Kₐ, which is defined as the molar concentration of protein-antibody complex divided by the molar concentrations of free antigen and free antibody under equilibrium conditions. The Kₐ determined for a preparation of polyclonal antibodies, which are heterogeneous in their affinities for multiple antigenic determinants, represents the average affinity, or avidity, of the antibodies. The Kₐ determined for a preparation of monoclonal antibodies, which are specific for a particular antigenic determinant, represents a true measure of affinity. High-affinity antibody preparations with Kₐ ranging from about 10⁹ to 10¹² L/mole are preferred for use in immunoassays in which the protein-antibody complex must withstand rigorous manipulations. Low-affinity antibody preparations with Kₐ ranging from about 10⁶ to 10⁷ L/mole are preferred for use in immunopurification and similar procedures which ultimately require dissociation of the protein, preferably in active form, from the antibody (Catty (1988) Antibodies, Volume I: A Practical Approach, IRL Press, Washington D.C.; Liddell and Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing about 5-10 mg specific antibody/ml, is generally employed in procedures requiring precipitation of protein-antibody complexes. Procedures for making antibodies, evaluating antibody specificity, titer, and avidity, and guidelines for antibody quality and usage in various applications, are widely available (Catty (supra); Ausubel (supra) pp. 11.1-11.31).

Diagnostics

Labeling of Molecules for Assay

A wide variety of reporter molecules and conjugation techniques are known by those skilled in the art and may be used in various nucleic acid, amino acid, and antibody assays. Synthesis of labeled molecules may be achieved using commercially available kits (Promega, Madison Wis.) for incorporation of a labeled nucleotide such as ³²P-dCTP (APB), Cy3-dCTP or Cy5-dCTP (Qiagen-Operon, Alameda Calif.), or amino acid such as ³⁵S-methionine (APB). Nucleotides and amino acids may be directly labeled with a variety of substances including fluorescent, chemiluminescent, or chromogenic agents, and the like, by chemical conjugation to amines, thiols and other groups present in the molecules using reagents such as BIODIPY or FITC (Molecular Probes).

Nucleic Acid Assays

The cDNAs, fragments, oligonucleotides, complementary RNA and nucleic acid molecules, and peptide nucleic acids may be used to detect and quantify differential gene expression for diagnosis of a disorder. Similarly antibodies which specifically bind LCAP may be used to quantitate the protein. Disorders associated with such differential expression particularly include cancers of the bladder, breast, colon, kidney, lung, lymph node, ovary and uterus and immune complications associated with these cancers. The diagnostic assay may use hybridization or amplification technology to compare gene expression in a biological sample from a patient to standard samples in order to detect differential gene expression. Qualitative or quantitative methods for this comparison are well known in the art.

Expression Profiles

A gene expression profile comprises the expression of a plurality of cDNAs as measured by after hybridization with a sample. The cDNAs of the invention may be used as elements on a array to produce a gene expression profile. In one embodiment, the array is used to diagnose or monitor the progression of disease. Researchers can assess and catalog the differences in gene expression between healthy and diseased tissues or cells.

For example, the cDNA or probe may be labeled by standard methods and added to a biological sample from a patient under conditions for the formation of hybridization complexes. After an incubation period, the sample is washed and the amount of label (or signal) associated with hybridization complexes, is quantified and compared with a standard value. If complex formation in the patient sample is significantly altered (higher or lower) in comparison to either a normal or disease standard, then differential expression indicates the presence of a disorder.

In order to provide standards for establishing differential expression, normal and disease expression profiles are established. This is accomplished by combining a sample taken from normal subjects, either animal or human, with a cDNA under conditions for hybridization to occur. Standard hybridization complexes may be quantified by comparing the values obtained using normal subjects with values from an experiment in which a known amount of a purified sequence is used. Standard values obtained in this manner may be compared with values obtained from samples from patients who were diagnosed with a particular condition, disease, or disorder. Deviation from standard values toward those associated with a particular disorder is used to diagnose that disorder.

By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, the array is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

In another embodiment, animal models which mimic a human disease can be used to characterize expression profiles associated with a particular condition, disease, or disorder; or treatment of the condition, disease, or disorder. Novel treatment regimens may be tested in these animal models using arrays to establish and then follow expression profiles over time. In addition, arrays may be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

Such assays may also be used to evaluate the efficacy of a particular therapeutic treatment regimen in animal studies or in clinical trials or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to years.

Protein Assays

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purifed, labeled standards are well known in the art (Ausubel, supra, unit 10.1-10.6). A two-site, monoclonal-based immunoassay utilizing antibodies reactive to two non-interfering epitopes is preferred, but a competitive binding assay may be employed (Pound (1998) Immunochemical Protocols, Humana Press, Totowa N.J.).

These methods are also useful for diagnosing diseases that show differential protein expression. Normal or standard values for protein expression are established by combining body fluids or cell extracts taken from a normal mammalian or human subject with specific antibodies to a protein under conditions for complex formation. Standard values for complex formation in normal and diseased tissues are established by various methods, often photometric means. Then complex formation as it is expressed in a subject sample is compared with the standard values. Deviation from the normal standard and toward the diseased standard provides parameters for disease diagnosis or prognosis while deviation away from the diseased and toward the normal standard may be used to evaluate treatment efficacy.

Recently, antibody arrays have allowed the development of techniques for high-throughput screening of recombinant antibodies. Such methods use robots to pick and grid bacteria containing antibody genes, and a filter-based ELISA to screen and identify clones that express antibody fragments. Because liquid handling is eliminated and the clones are arrayed from master stocks, the same antibodies can be spotted multiple times and screened against multiple antigens simultaneously. Antibody arrays are useful in the identification of differentially expressed proteins. (See de Wildt et al. (2000) Nat Biotechnol 18:989-94.)

Differential expression of LCAP as detected using the cDNA encoding LCAP, LCAP or an antibody that specifically binds LCAP and any of the above assays can be used to diagnose non-small cell lung cancer, cancers of the bladder, breast, colon, kidney, lymph node, ovary and uterus, and any immune complication associated with any of these cancers.

Therapeutics

Chemical and structural homology exits among LCAP (SEQ ID NO:1), and human and pig cathepsins (SEQ ID NO:18 and SEQ ID NO:19, respectively). Expression of LCAP is associated with cell proliferation, particularly in cDNA libraries associated with cancer, immune disorders, and fetal/infant development. As shown in FIG. 3, differential expression of LCAP is associated with cancers of the bladder, breast, colon, kidney, lymph node, ovary and uterus and, in particular, non-small cell lung cancers.

In one embodiment, an antagonist of LCAP, or a fragment or a derivative thereof, may be administered to a subject to prevent or treat a disorder associated with cell proliferation. Disorders of cell proliferation include various types of cancer including, but not limited to, adenocarcinoma, sarcoma, lymphoma, leukemia, melanoma, myeloma, teratocarcinoma. These include cancers of the adrenal gland, bladder, bone, brain, breast, colon, esophagus, heart, kidney, liver, lymph node, ovary, pancreas, paraganglia, parathyroid, prostate, salivary glands, skin, spleen, stomach, testis, thyroid, and uterus, and in particular, lung cancer. In one aspect, an antibody specific for LCAP may be used directly as an antagonist, or indirectly as a targeting or delivery mechanism for bringing a pharmaceutical agent to cells or tissue which express LCAP.

In another embodiment, a vector expressing the complement of the polynucleotide encoding LCAP, or a fragment or a derivative thereof, may be administered to a subject to prevent or treat a cancer including, but not limited to, those described above.

In another embodiment, an antagonist of LCAP or a fragment or a derivative thereof, may be administered to a subject to prevent or treat an immune disorder. Such disorders include, but are not limited to, Addison's disease, AIDS, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polycystic kidney disease, polymyositis, rheumatoid arthritis, scleroderma, Sjogren's syndrome, autoimmune thyroiditis. In one aspect, an antibody specific for LCAP may be used directly as an antagonist, or indirectly as a targeting or delivery mechanism for bringing a pharmaceutical agent to cells or tissue which express LCAP.

In one embodiment, when decreased expression or activity of the protein is desired, an inhibitor, antagonist, antibody and the like or a pharmaceutical agent containing one or more of these molecules may be delivered. Such delivery may be effected by methods well known in the art and may include delivery by an antibody specifically targeted to the protein. Neutralizing antibodies which inhibit dimer formation are generally preferred for therapeutic use.

In another embodiment, when increased expression or activity of the protein is desired, the protein, an agonist, an enhancer and the like or a pharmaceutical agent containing one or more of these molecules may be delivered. Such delivery may be effected by methods well known in the art and may include delivery of a pharmaceutical agent by an antibody specifically targeted to the protein.

Any of the cDNAs, complementary molecules, or fragments thereof, proteins or portions thereof, vectors delivering these nucleic acid molecules or expressing the proteins, and their ligands may be administered in combination with other therapeutic agents. Selection of the agents for use in combination therapy may be made by one of ordinary skill in the art according to conventional pharmaceutical principles. A combination of therapeutic agents may act synergistically to affect treatment of a particular disorder at a lower dosage of each agent.

Modification of Gene Expression Using Nucleic Acids

Gene expression may be modified by designing complementary or antisense molecules (DNA, RNA, or PNA) to the control, 5′, 3′, or other regulatory regions of the gene encoding LCAP. Oligonucleotides designed to inhibit transcription initiation are preferred. Similarly, inhibition can be achieved using triple helix base-pairing which inhibits the binding of polymerases, transcription factors, or regulatory molecules (Gee et al. In: Huber and Carr (1994) Molecular and Immunologic Approaches, Futura Publishing, Mt. Kisco N.Y., pp. 163-177). A complementary molecule may also be designed to block translation by preventing binding between ribosomes and mRNA. In one alternative, a library or plurality of cDNAs may be screened to identify those which specifically bind a regulatory, nontranslated sequence. These libraries or pluralities of molecule include artificial chromosome constructions, antisense molecules, DNA molecules, peptide nucleic acids, peptides, proteins, regulatory molecules, RNA molecules, rhibozymes, repressors, and transcription factors.

Ribozymes, enzymatic RNA molecules, may also be used to catalyze the specific cleavage of RNA. The mechanism of ribozyme action involves sequence-specific hybridization of the ribozyme molecule to complementary target RNA followed by endonucleolytic cleavage at sites such as GUA, GUU, and GUC. Once such sites are identified, an oligonucleotide with the same sequence may be evaluated for secondary structural features which would render the oligonucleotide inoperable. The suitability of candidate targets may also be evaluated by testing their hybridization with complementary oligonucleotides using ribonuclease protection assays.

Complementary nucleic acids and ribozymes of the invention may be prepared via recombinant expression, in vitro or in vivo, or using solid phase phosphoramidite chemical synthesis. In addition, RNA molecules may be modified to increase intracellular stability and half-life by addition of flanking sequences at the 5′ and/or 3′ ends of the molecule or by the use of phosphorothioate or 2′ O-methyl rather than phosphodiesterase linkages within the backbone of the molecule. Modification is inherent in the production of PNAs and can be extended to other nucleic acid molecules. Either the inclusion of nontraditional bases such as inosine, queosine, and wybutosine, or the modification of adenine, cytidine, guanine, thymine, and uridine with acetyl-, methyl-, thio- groups renders the molecule less available to endogenous endonucleases.

cDNA Therapeutics

The cDNAs of the invention can be used in gene therapy. cDNAs can be delivered ex vivo to target cells, such as cells of bone marrow. Once stable integration and transcription and or translation are confirmed, the bone marrow may be reintroduced into the subject. Expression of the protein encoded by the cDNA may correct a disorder associated with mutation of a normal sequence, reduction or loss of an endogenous target protein, or overepression of an endogenous or mutant protein. Alternatively, cDNAs may be delivered in vivo using vectors such as retrovirus, adenovirus, adeno-associated virus, herpes simplex virus, and bacterial plasmids. Non-viral methods of gene delivery include cationic liposomes, polylysine conjugates, artificial viral envelopes, and direct injection of DNA (Anderson (1998) Nature 392:25-30; Dachs et al. (1997) Oncol Res 9:313-325; Chu et al. (1998) J Mol Med 76(3-4):184-192; Weiss et al. (1999) Cell Mol Life Sci 55(3):334-358; Agrawal (1996) Antisense Therapeutics, Humana Press, Totowa N.J.; and August et al. (1997) Gene Therapy (Advances in Pharmacology, Vol. 40), Academic Press, San Diego Calif.).

Screening and Purification Assays

The cDNA encoding LCAP may be used to screen a library or a plurality of molecules or compounds for specific binding affinity. The libraries may be artificial chromosome constructions, antisense molecules, branched nucleic acid molecules, DNA molecules, RNA molecules, peptide nucleic acids, peptides, proteins such as transcription factors, enhancers, or repressors, ribozymes and other ligands which regulate the activity, replication, transcription, or translation of the endogenous gene. The assay involves combining a polynucleotide with a library or plurality of molecules or compounds under conditions allowing specific binding, and detecting specific binding to identify at least one molecule which specifically binds the single-stranded or double-stranded molecule.

In one embodiment, the cDNA of the invention may be incubated with a plurality of purified molecules or compounds and binding activity determined by methods well known in the art, e.g., a gel-retardation assay (U.S. Pat. No. 6,010,849) or a reticulocyte lysate transcriptional assay. In another embodiment, the cDNA may be incubated with nuclear extracts from biopsied and/or cultured cells and tissues. Specific binding between the cDNA and a molecule or compound in the nuclear extract is initially determined by gel shift assay and may be later confirmed by recovering and raising antibodies against that molecule or compound. When these antibodies are added into the assay, they cause a supershift in the gel-retardation assay.

In another embodiment, the cDNA may be used to purify a molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and collected.

In a further embodiment, the protein or a portion thereof may be used to purify a ligand from a sample. A method for using a protein to purify a ligand would involve combining the protein with a sample under conditions to allow specific binding, detecting specific binding between the protein and ligand, recovering the bound protein, and using a chaotropic agent to separate the protein from the purified ligand.

In a preferred embodiment, LCAP may be used to screen a plurality of molecules or compounds in any of a variety of screening assays. The portion of the protein employed in such screening may be free in solution, affixed to an abiotic or biotic substrate (e.g. borne on a cell surface), or located intracellularly. For example, in one method, viable or fixed prokaryotic host cells that are stably transformed with recombinant nucleic acids that have expressed and positioned a peptide on their cell surface can be used in screening assays. The cells are screened against a plurality or libraries of ligands, and the specificity of binding or formation of complexes between the expressed protein and the ligand can be measured. Depending on the particular kind of molecules or compounds being screened, the assay may be used to identify agonists, antagonists, antibodies, DNA molecules, small drug molecules, immunoglobulins, inhibitors, mimetics, peptide nucleic acids, peptides, proteins, and RNA molecules or any other ligand, which specifically binds the protein.

In one aspect, this invention comtemplates a method for high throughput screening using very small assay volumes and very small amounts of test compound as described in U.S. Pat. No. 5,876,946, incorporated herein by reference. This method is used to screen large numbers of molecules and compounds via specific binding. In another aspect, this invention also contemplates the use of competitive drug screening assays in which neutralizing antibodies capable of binding the protein specifically compete with a test compound capable of binding to the protein. Molecules or compounds identified by screening may be used in a mammalian model system to evaluate their toxicity, diagnostic, or therapeutic potential.

Pharmaceutical Compositions

Pharmaceutical compositions may be formulated and administered, to a subject in need of such treatment, to attain a therapeutic effect. Such compositions contain the instant protein, agonists, antibodies specifically binding the protein, antagonists, inhibitors, or mimetics of the protein. Compositions may be manufactured by conventional means such as mixing, dissolving, granulating, dragee-making, levigating, emulsifying, encapsulating, entrapping, or lyophilizing. The composition may be provided as a salt, formed with acids such as hydrochloric, sulfuric, acetic, lactic, tartaric, malic, and succinic, or as a lyophilized powder which may be combined with a sterile buffer such as saline, dextrose, or water. These compositions may include auxiliaries or excipients which facilitate processing of the active compounds.

Auxiliaries and excipients may include coatings, fillers or binders including sugars such as lactose, sucrose, mannitol, glycerol, or sorbitol; starches from corn, wheat, rice, or potato; proteins such as albumin, gelatin and collagen; cellulose in the form of hydroxypropylmethyl-cellulose, methyl cellulose, or sodium carboxymethylcellulose; gums including arabic and tragacanth; lubricants such as magnesium stearate or talc; disintegrating or solubilizing agents such as the, agar, alginic acid, sodium alginate or cross-linked polyvinyl pyrrolidone; stabilizers such as carbopol gel, polyethylene glycol, or titanium dioxide; and dyestuffs or pigments added for identify the product or to characterize the quantity of active compound or dosage.

These compositions may be administered by any number of routes including oral, intravenous, intramuscular, intra-arterial, intramedullary, intrathecal, intraventricular, transdermal, subcutaneous, intraperitoneal, intranasal, enteral, topical, sublingual, or rectal.

The route of administration and dosage will determine formulation; for example, oral administration may be accomplished using tablets, pills, dragees, capsules, liquids, gels, syrups, slurries, or suspensions; parenteral administration may be formulated in aqueous, physiologically compatible buffers such as Hanks' solution, Ringer's solution, or physiologically buffered saline. Suspensions for injection may be aqueous, containing viscous additives such as sodium carboxymethyl cellulose or dextran to increase the viscosity, or oily, containing lipophilic solvents such as sesame oil or synthetic fatty acid esters such as ethyl oleate or triglycerides, or liposomes. Penetrants well known in the art are used for topical or nasal administration.

Toxicity and Therapeutic Efficacy

A therapeutically effective dose refers to the amount of active ingredient which ameliorates symptoms or condition. For any compound, a therapeutically effective dose can be estimated from cell culture assays using normal and neoplastic cells or in animal models. Therapeutic efficacy, toxicity, concentration range, and route of administration may be determined by standard pharmaceutical procedures using experimental animals.

The therapeutic index is the dose ratio between therapeutic and toxic effects—LD50 (the dose lethal to 50% of the population)/ED50 (the dose therapeutically effective in 50% of the population)—and large therapeutic indices are preferred. Dosage is within a range of circulating concentrations, includes an ED50 with little or no toxicity, and varies depending upon the composition, method of delivery, sensitivity of the patient, and route of administration. Exact dosage will be determined by the practitioner in light of factors related to the subject in need of the treatment.

Dosage and administration are adjusted to provide active moiety that maintains therapeutic effect. Factors for adjustment include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular composition.

Normal dosage amounts may vary from 0.1 μg, up to a total dose of about 1 g, depending upon the route of administration. The dosage of a particular composition may be lower when administered to a patient in combination with other agents, drugs, or hormones. Guidance as to particular dosages and methods of delivery is provided in the pharmaceutical literature and generally available to practitioners. Further details on techniques for formulation and administration may be found in the latest edition of Remington's Pharmaceutical Sciences (Mack Publishing, Easton Pa.).

Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of under- or over-expression of genes of interest and for the development of methods for diagnosis and treatment of diseases. A mammal inbred to over-express a particular gene (for example, secreted in milk) may also serve as a convenient source of the protein expressed by that gene.

Toxicology

Toxicology is the study of the effects of agents on living systems. The majority of toxicity studies are performed on rats or mice. Observation of qualitative and quantitative changes in physiology, behavior, homeostatic processes, and lethality in the rats or mice are used to generate a toxicity profile and to assess potential consequences on human health following exposure to the agent.

Genetic toxicology identifies and analyzes the effect of an agent on the rate of endogenous, spontaneous, and induced genetic mutations. Genotoxic agents usually have common chemical or physical properties that facilitate interaction with nucleic acids and are most harmful when chromosomal aberrations are transmitted to progeny. Toxicological studies may identify agents that increase the frequency of structural or functional abnormalities in the tissues of the progeny if administered to either parent before conception, to the mother during pregnancy, or to the developing organism. Mice and rats are most frequently used in these tests because their short reproductive cycle allows the production of the numbers of organisms needed to satisfy statistical requirements.

Acute toxicity tests are based on a single administration of an agent to the subject to determine the symptomology or lethality of the agent. Three experiments are conducted: 1) an initial dose-range-finding experiment, 2) an experiment to narrow the range of effective doses, and 3) a final experiment for establishing the dose-response curve.

Subchronic toxicity tests are based on the repeated administration of an agent. Rat and dog are commonly used in these studies to provide data from species in different families. With the exception of carcinogenesis, there is considerable evidence that daily administration of an agent at high-dose concentrations for periods of three to four months will reveal most forms of toxicity in adult animals.

Chronic toxicity tests, with a duration of a year or more, are used to demonstrate either the absence of toxicity or the carcinogenic potential of an agent. When studies are conducted on rats, a minimum of three test groups plus one control group are used, and animals are examined and monitored at the outset and at intervals throughout the experiment.

Transgenic Animal Models

Transgenic rodents that over-express or under-express a gene of interest may be inbred and used to model human diseases or to test therapeutic or toxic agents. (See, e.g., U.S. Pat. No. 5,175,383 and U.S. Pat. No. 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Embryonic Stem Cells

Embryonic (ES) stem cells isolated from rodent embryos retain the potential to form embryonic tissues. When ES cells are placed inside a carrier embryo, they resume normal development and contribute to tissues of the live-born animal. ES cells are the preferred cells used in the creation of experimental knockout and knockin rodent strains. Mouse ES cells, such as the mouse 129/SvJ cell line, are derived from the early mouse embryo and are grown under culture conditions well known in the art. Vectors used to produce a transgenic strain contain a disease gene candidate and a marker gene, the latter serves to identify the presence of the introduced disease gene. The vector is transformed into ES cells by methods well known in the art, and transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains.

ES cells derived from human blastocysts may be manipulated in vitro to differentiate into at least eight separate cell lineages. These lineages are used to study the differentiation of various cell types and tissues in vitro, and they include endoderm, mesoderm, and ectodermal cell types which differentiate into, for example, neural cells, hematopoietic lineages, and cardiomyocytes.

Knockout Analysis

In gene knockout analysis, a region of a gene is enzymatically modified to include a non-mammalian gene such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene. Transformed cells are injected into rodent blastulae, and the blastulae are implanted into pseudopregnant dams. Transgenic progeny are crossbred to obtain homozygous inbred lines which lack a functional copy of the mammalian gene. In one example, the mammalian gene is a human gene.

Knockin Analysis

ES cells can be used to create knockin humanized animals (pigs) or transgenic animal models (mice or rats) of human diseases. With knockin technology, a region of a human gene is injected into animal ES cells, and the human sequence integrates into the animal cell genome. Transformed cells are injected into blastulae and the blastulae are implanted as described above. Transgenic progeny or inbred lines are studied and treated with potential pharmaceutical agents to obtain information on treatment of the analogous human condition. These methods have been used to model several human diseases.

Non-Human Primate Model

The field of animal testing deals with data and methodology from basic sciences such as physiology, genetics, chemistry, pharmacology and statistics. These data are paramount in evaluating the effects of therapeutic agents on non-human primates as they can be related to human health. Monkeys are used as human surrogates in vaccine and drug evaluations, and their responses are relevant to human exposures under similar conditions. Cynomolgus and Rhesus monkeys (Macaca fascicularis and Macaca mulatta, respectively) and Common Marmosets (Callithrix jacchus) are the most common non-human primates (NHPs) used in these investigations. Since great cost is associated with developing and maintaining a colony of NHPs, early research and toxicological studies are usually carried out in rodent models. In studies using behavioral measures such as drug addiction, NHPs are the first choice test animal. In addition, NHPs and individual humans exhibit differential sensitivities to many drugs and toxins and can be classified as a range of phenotypes from “extensive metabolizers” to “poor metabolizers” of these agents.

In additional embodiments, the cDNAs which encode the protein may be used in any molecular biology techniques that have yet to be developed, provided the new techniques rely on properties of cDNAs that are currently known, including, but not limited to, such properties as the triplet genetic code and specific base pair interactions.

### EXAMPLES

I cDNA Library Construction

The THYMNOT02 cDNA library was constructed using polyA RNA isolated from the thymus tissue removed from a 3-year old Caucasian male. First strand cDNA synthesis was accomplished using an oligo d(T) primer/linker which also contained an XhoI restriction site. Second strand synthesis was performed using a combination of DNA polymerase I, E. coli ligase, and RNAse H, followed by the addition of an EcoRI adaptor to the blunt ended cDNA. The EcoRi adapted, double-stranded cDNA was digested with XhoI restriction enzyme to obtain sequences which were inserted into the UNIZAP vector system (Stratagene). The vector which contained the pBLUESCRIPT phagemid was transformed into competent E. coli host cells, strain XL1-BlueMRF (Stratagene).

The phagemid forms of individual cDNA clones were obtained by the in vivo excision process. Enzymes from both pBluescript and a cotransformed f1 helper phage nicked the DNA, initiated new DNA synthesis, and created the smaller, single-stranded circular phagemid molecules which contained the cDNA insert. The phagemid DNA was released, purified, and used to reinfect fresh SOLR host cells (Stratagene). Presence of the phagemid which carries the gene for β-lactamase allowed transformed bacteria to grow on medium containing ampicillin.

II Isolation and Sequencing of cDNA Clones

Plasmid DNA was released from the cells and purified using the MINIPREP kit and recommended protocol (Advanced Genetic Technologies Corporation, Gaithersburg Md.) except for the following changes: 1) the 96 wells were each filled with only 1 ml of sterile TERRIFIC BROTH (BD Biosciences) with carbenicillin at 25 mg/L and glycerol at 0.4%; 2) after inoculation, the bacteria were cultured for 24 hours and then lysed with 60 μl of lysis buffer; 3) the block was centrifuged @2900 rpm for 5 min in the GS-6R (Beckman Coulter, Fullerton Calif.) before the contents of the block were added to the primary filter plate; and 4) the optional step of adding isopropanol to TRIS buffer was not routinely performed. After the last step in the protocol, samples were transferred to a 96-well block for storage.

Alternative methods of purifying plasmid DNA include the use of MAGIC MINIPREPS DNA purification system (Promega, Madison Wis.) or QIAWELL8 plasmid, QIAWELL PLUS and QIAWELL ULTRA DNA purification systems (Qiagen, Chatsworth Calif.).

The cDNAs were prepared using a MICROLAB 2200 (Hamilton, Reno Nev.) in combination with DNA ENGINE thermal cyclers (MJ Research) and sequenced by the method of Sanger and Coulson (1975; J Mol Biol 94:441-48) using PRISM 377 or 373 DNA sequencing systems (ABI). Reading frame was determined using standard techniques.

The nucleotide sequences and/or amino acid sequences of the Sequence Listing were used to query sequences in the GenBank, SwissProt, BLOCKS, and Pima II databases. BLAST produced alignments of both nucleotide and amino acid sequences to determine sequence similarity. Because of the local nature of the alignments, BLAST was especially useful in determining exact matches or in identifying homologs which may be of prokaryotic (bacterial) or eukaryotic (animal, fungal, or plant) origin. Other algorithms such as those of Smith et al. (1992; Protein Engineering 5:35-51) could have been used when dealing with primary sequence patterns and secondary structure gap penalties. The sequences disclosed in this application have lengths of at least 49 nucleotides and have no more than 12% uncalled bases (where N is recorded rather than A, C, G, or T).

The BLAST approach searched for matches between a query sequence and a database sequence. BLAST evaluated the statistical significance of any matches found, and reported only those matches that satisfy the user-selected threshold of significance. In this application, threshold was set at 10⁻²⁵ for nucleotides and 10⁻¹⁰ for peptides.

IV Extension of cDNAs

The cDNAs were extended using the cDNA clone and oligonucleotide primers. One primer was synthesized to initiate 5′ extension of the known fragment, and the other, to initiate 3′ extension of the known fragment. The initial primers were designed using commercially available primer analysis software to be about 22 to 30 nucleotides in length, to have a GC content of about 50% or more, and to anneal to the target sequence at temperatures of about 68C to about 72C. Any stretch of nucleotides that would result in hairpin structures and primer-primer dimerizations was avoided.

Selected cDNA libraries were used as templates to extend the sequence. If more than one extension was necessary, additional or nested sets of primers were designed. Preferred libraries have been size-selected to include larger cDNAs and random primed to contain more sequences with 5′ or upstream regions of genes. Genomic libraries are used to obtain regulatory elements, especially extension into the 5′ promoter binding region.

High fidelity amplification was obtained by PCR using methods such as that taught in U.S. Pat. No. 5,932,451. PCR was performed in 96-well plates using the DNA ENGINE thermal cycler (MJ Research). The reaction mix contained DNA template, 200 nmol of each primer, reaction buffer containing Mg²+, (NH₄)₂SO₄, and β-mercaptoethanol, Taq DNA polymerase (APB), ELONGASE enzyme (Invitrogen), and Pfu DNA polymerase (Stratagene), with the following parameters for primer pair PCI A and PCI B (Incyte Genomics): Step 1: 94C, three min; Step 2: 94C, 15 sec; Step 3: 60C, one min; Step 4: 68C, two min; Step 5: Steps 2, 3, and 4 repeated 20 times; Step 6: 68C, five min; and Step 7: storage at 4C. In the alternative, the parameters for primer pair T7 and SK+ (Stratagene) were as follows: Step 1: 94C, three min; Step 2: 94C, 15 sec; Step 3: 57C, one min; Step 4: 68C, two min; Step 5: Steps 2, 3, and 4 repeated 20 times; Step 6: 68C, five min; and Step 7: storage at 4C.

The concentration of DNA in each well was determined by dispensing 100 μl PICOGREEN quantitation reagent (0.25% reagent in 1× TE, v/v; Molecular Probes) and 0.5 μl of undiluted PCR product into each well of an opaque fluorimeter plate (Corning Life Sciences, Acton Mass.) and allowing the DNA to bind to the reagent. The plate was scanned in a Fluoroskan II (Labsystems Oy, Helsinki Finland) to measure the fluorescence of the sample and to quantify the concentration of DNA. A 5 μl to 10 μl aliquot of the reaction mixture was analyzed by electrophoresis on a 1% agarose minigel to determine which reactions were successful in extending the sequence.

The extended clones were desalted, concentrated, transferred to 384-well plates, digested with CviJI cholera virus endonuclease (Molecular Biology Research, Madison Wis.), and sonicated or sheared prior to religation into pUC18 vector (APB). For shotgun sequences, the digested nucleotide sequences were separated on low concentration (0.6 to 0.8%) agarose gels, fragments were excised, and the agar was digested with AGARACE enzyme (Promega). Extended clones were religated using T4 DNA ligase (New England Biolabs) into pUC18 vector (APB), treated with Pfu DNA polymerase (Stratagene) to fill-in restriction site overhangs, and transfected into E. coli competent cells. Transformed cells were selected on antibiotic-containing media, and individual colonies were picked and cultured overnight at 37C in 384-well plates in LB/2× carbenicillin liquid media.

The cells were lysed, and DNA was amplified using primers, Taq DNA polymerase (APB) and Pfu DNA polymerase (Stratagene) with the following parameters: Step 1: 94C, three min; Step 2: 94C, 15 sec; Step 3: 60C, one min; Step 4: 72C, two min; Step 5: steps 2, 3, and 4 repeated 29 times; Step 6: 72C, five min; and Step 7: storage at 4C. DNA was quantified using PICOGREEN quantitation reagent (Molecular Probes) as described above. Samples with low DNA recoveries were reamplified using the conditions described above. Samples were diluted with 20% dimethylsulfoxide (DMSO; 1:2, v/v), and sequenced using DYENAMIC energy transfer sequencing primers and the DYENAMIC DIRECT cycle sequencing kit (APB) or the PRISM BIGDYE terminator cycle sequencing kit (ABI).

IV Homology Searching of cDNA Clones and Their Deduced Proteins

The cDNAs of the Sequence Listing or their deduced amino acid sequences were used to query databases such as GenBank, SwissProt, BLOCKS, and the like. These databases that contain previously identified and annotated sequences or domains were searched using BLAST or BLAST2 to produce alignments and to determine which sequences were exact matches or homologs. The alignments were to sequences of prokaryotic (bacterial) or eukaryotic (animal, fungal, or plant) origin. Alternatively, algorithms such as the one described in Smith and Smith (1992, Protein Engineering 5:35-51) could have been used to deal with primary sequence patterns and secondary structure gap penalties. All of the sequences disclosed in this application have lengths of at least 49 nucleotides, and no more than 12% uncalled bases (where N is recorded rather than A, C, G, or T).

As detailed in Karlin and Altschul (1993; Proc Natl Acad Sci 90:5873-5877), BLAST matches between a query sequence and a database sequence were evaluated statistically and only reported when they satisfied the threshold of 10⁻²⁵ for nucleotides and 10⁻¹⁴ for peptides. Homology was also evaluated by product score calculated as follows: the % nucleotide or amino acid identity [between the query and reference sequences] in BLAST is multiplied by the % maximum possible BLAST score [based on the lengths of query and reference sequences] and then divided by 100. In comparison with hybridization procedures used in the laboratory, the stringency for an exact match was set from a lower limit of about 40 (with 1-2% error due to uncalled bases) to a 100% match of about 70.

The BLAST software suite (NCBI, Bethesda Md.), includes various sequence analysis programs including “blastn” that is used to align nucleotide sequences and BLAST2 that is used for direct pairwise comparison of either nucleotide or amino acid sequences. BLAST programs are commonly used with gap and other parameters set to default settings, e.g.: Matrix: BLOSUM62; Reward for match: 1; Penalty for mismatch: −2; Open Gap: 5 and Extension Gap: 2 penalties; Gap x drop-off: 50; Expect: 10; Word Size: 11; and Filter: on. Identity is measured over the entire length of a sequence. Brenner et al. (1998; Proc Natl Acad Sci 95:6073-6078, incorporated herein by reference) analyzed BLAST for its ability to identify structural homologs by sequence identity and found 30% identity is a reliable threshold for sequence alignments of at least 150 residues and 40%, for alignments of at least 70 residues.

The cDNAs of this application were compared with assembled consensus sequences or templates found in the LIFESEQ GOLD database (Incyte Genomics). Component sequences from cDNA, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another, and those having local similarity of at least 82% were combined and reassembled. Bins having templates with less than 95% local identity were split. Templates were subjected to analysis by STITCHER/EXON MAPPER algorithms that determine the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homolog match as having an E-value (or probability score) of ≦1×10⁻⁸. The templates were also subjected to frameshift FASTx against GENPEPT, and homolog match was defined as having an E-value of ≦1×10⁻⁸. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. No. 08/812,290 and U.S. Ser. No. 08/811,758, both filed Mar. 6, 1997; in U.S. Ser. No. 08/947,845, filed Oct. 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis Mo.). The cDNA was further analyzed using MACDNASIS PRO software (Hitachi Software Engineering), and LASERGENE software (DNASTAR) and queried against public databases such as the GenBank rodent, mammalian, vertebrate, prokaryote, and eukaryote databases, SwissProt, BLOCKS, PRINTS, PFAM, and Prosite.

V Northern Analysis and Transcript Imaging

Northern analysis is a laboratory technique well known in the art which is used to detect the presence of a transcript of a gene and involves the hybridization of a labeled nucleotide sequence to a membrane on which RNAs from a particular cell type or tissue have been bound. It is described in Sambrook, supra, ch. 7 and Ausubel et al. (1989) Current Protocols in Molecular Biology, John Wiley & Sons, New York N.Y., ch. 4 and 16, incorporated herein by reference).

Analogous computer techniques applying BLAST are used to search for identical or related molecules in nucleotide databases such as GenBank or the LIFESEQ database (Incyte Genomics). This analysis is faster than multiple membrane-based hybridizations. In addition, the sensitivity of the computer search can be modified to determine whether any particular match is categorized as exact or homologous. The basis of the search is the product score, which is defined as:

$$\frac{\% \text{sequence identity} \times \% \text{maximum BLAST score}}{100}$$

The product score takes into account both the degree of similarity between two sequences and the length of the sequence match. For example, with a product score of 40, the match will be exact within a 1% to 2% error, and, with a product score of 70, the match will be exact. Homologous molecules are usually identified by selecting those which show product scores between 15 and 40, although lower scores may identify related molecules.

The results of northern analysis were summarized in THE INVENTION section as disease areas and percent abundance in which the transcript encoding LCAP occurs. Abundance directly reflects the number of times a particular transcript is represented in a cDNA library, and percent abundance is abundance divided by the total number of sequences examined in the cDNA library.

Transcript Imaging

A transcript image is performed using the LIFESEQ GOLD database (Incyte Genomics). This process allows assessment of the relative abundance of the expressed polynucleotides in all cDNA libraries in the database and was described in U.S. Pat. No. 5,840,484, incorporated herein by reference. All sequences and cDNA libraries are categorized by system, organ/tissue and cell type. The categories include cardiovascular system, connective tissue, digestive system, embryonic structures, endocrine system, exocrine glands, female and male genitalia, germ cells, hemic/immune system, liver, musculoskeletal system, nervous system, pancreas, respiratory system, sense organs, skin, stomatognathic system, unclassified/mixed, and the urinary tract. Criteria for transcript imaging are selected from category, number of cDNAs per library, library description, disease indication, clinical relevance of sample, and the like.

For each category, the number of libraries in which the sequence was expressed are counted and shown over the total number of libraries in that category. For each library, the number of cDNAs are counted and shown over the total number of cDNAs in that library. In some transcript images, all enriched, normalized or subtracted libraries, which have high copy number sequences can be removed prior to processing, and all mixed or pooled tissues, which are considered non-specific in that they contain more than one tissue type or more than one subject's tissue, can be excluded from the analysis. Treated and untreated cell lines and/or fetal tissue data can also be excluded where clinical relevance is emphasized. Conversely, fetal tissue can be emphasized wherever elucidation of inherited disorders or differentiation of particular adult or embryonic stem cells into tissues or organs such as heart, kidney, nerves or pancreas would be aided by removing clinical samples from the analysis. Transcript imaging can also be used to support data from other methodologies such as guilt-by-association and hybridization technologies.

The results of TI are shown in the table below. The first column shows library name; the second column, the number of cDNAs sequenced in that library; the third column, the description of the library; the fourth column, absolute abundance of the transcript in the library; and the fifth column, percentage abundance of the transcript in the library.

Category: Respiratory (Lung)

| | | Description | | % |
|---|---|---|---|---|
| Library | cDNAs | of Tissue | Abundance | Abundance |
| LUNGTUP11 | 3692 | squamous cell | 1 | 0.0271 |
| | | CA, pool, 3′, | | |
| | | CGAP | | |
| LUNGTUP06 | 6207 | squamous cell | 1 | 0.0161 |
| | | CA, pooled, | | |
| | | NORM, CGAP | | |
| LUNGNOT30 | 8170 | lung, aw/Patau's, | 1 | 0.0122 |
| | | fetal, 20 wM | | |

SEQ ID NO:2 was expressed in the squamous cell carcinomas shown above and in a single fetal lung library. SEQ ID NO:2 was not expressed in asthmatic lung (LUNGAST01, LUNGNOT33, and LUNGNOT39), idiopathic pulmonary disease (LUNGDIN02, LUNGDIS03), cytologically normal lung (LUNGNOE02, LUNGNOF03, LUNGNOM01, LUNGNON03,LUNGNON07, LUNGNOP01, LUNGNOP03, LUNGNOP04, LUNGNOT01, LUNGNOT02, LUNGNOT03, LUNGNOT04, LUNGNOT09, LUNGNOT10, LUNGNOT12, LUNGNOT14,LUNGNOT18, LUNGNOT22, LUNGNOT23, LUNGNOT25, LUNGNOT27, LUNGNOT28, LUNGNOT31, LUNGNOT34, LUNGNOT35, LUNGNOT37, LUNGNOT38, LUNGNOT40, LUNGTMC01, LUNGTMT03, and LUNGTMT04), pneumonitis (LUNGNOT15), panacinar emphysema (LUNGNOT20), any other lung tumors or fetal lung libraries (LUNGFEC03, LUNGFEM01, LUNGFEN02, LUNGFEP01, LUNGFEP02, LUNGFER04, LUNGFET03, LUNGFET04, and LUNGFET05). When used in a clinically relevant and tissue specific manner, the expression of LCAP is diagnostic of lung cancer.

VI Chromosome Mapping

Radiation hybrid and genetic mapping data available from public resources such as the Stanford Human Genome Center (SHGC), Whitehead Institute for Genome Research (WIGR), and Généthon are used to determine if any of the cDNAs presented in the Sequence Listing have been mapped. Any of the fragments of the cDNA encoding LCAP that have been mapped result in the assignment of all related regulatory and coding sequences to the same location. The genetic map locations are described as ranges, or intervals, of human chromosomes. The map position of an interval, in cM (which is roughly equivalent to 1 megabase of human DNA), is measured relative to the terminus of the chromosomal p-arm.

VII Hybridization and Amplication Technologies and Analyses

Tissue Sample Preparation

The normal tissues were the Human Total RNA Master Panel (Clontech Laboratories) and human stomach poly-A (Clontech Laboratories).

Matched normal and cancerous lung tissue samples were provided by the Roy Castle International Centre for Lung Cancer Research (Liverpool UK) and are described in the table below.

| Donor | Tissue |
|---|---|
| 7173 | Tumor, right, squamous cell carcinoma |
| 7175 | Tumor, right upper lobe, adenocarcinoma |
| 7176 | Tumor, right middle, adenosquamous carcinoma |
| 7178 | Tumor, left upper lobe, squamous cell carcinoma |
| 7188 | Tumor, right upper lobe, adenocarcinoma |
| 9752 | Tumor, right lung, squamous cell carcinoma |
| 9757 | Tumor, right, adenocarcinoma |
| 9758 | Tumor, right upper, adenocarcinoma |
| 9760 | Tumor, right lung, squamous cell carcinoma |
| 9761 | Tumor, left lung, squamous cell carcinoma |
| 9762 | Tumor, right upper, adenocarcinoma |
| 9763 | Tumor, right lung, squamous cell carcinoma |
| 9764 | Tumor, right upper, adenocarcinoma |
| 9765 | Tumor, left lung, squamous cell carcinoma |

Immobilization of cDNAs on a Substrate

The cDNAs are applied to a substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37C for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning Life Sciences) by ultrasound in 0.1% SDS and acetone, etching in 4% hydrofluoric acid (VWR Scientific Products, West Chester Pa.), coating with 0.05% aminopropyl silane (Sigma Aldrich) in 95% ethanol, and curing in a 1° C. oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford Mass.) for 30 min at 60C; then the arrays are washed in 0.2% SDS and rinsed in distilled water as before.

Probe Preparation for Membrane Hybridization

Hybridization probes derived from the cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 100C for five min, and briefly centrifuging. The denatured cDNA is then added to a REDIPRIME tube (APB), gently mixed until blue color is evenly distributed, and briefly centrifuged. Five μl of [³²P]dCTP is added to the tube, and the contents are incubated at 37C for 10 min. The labeling reaction is stopped by adding 5 μl of 0.2M EDTA, and probe is purified from unincorporated nucleotides using a PROBEQUANT G-50 microcolumn (APB). The purified probe is heated to 100C for five min, snap cooled for two min on ice, and used in membrane-based hybridizations as described below.

Probe Preparation for QPCR

Probes for the QPCR analysis were prepared according to the standard TAQMAN protocol (ABI).

Probe Preparation for Polymer Coated Slide Hybridization

Hybridization probes derived from mRNA isolated from samples are employed for screening cDNAs of the Sequence Listing in array-based hybridizations. Probe is prepared using the GEMbright kit (Incyte Genomics) by diluting mRNA to a concentration of 200 ng in 9 μl TE buffer and adding 5 μl 5× buffer, 1 μl 0.1 M DTT, 3 μl Cy3 or Cy5 labeling mix, 1 μl RNAse inhibitor, 1 μl reverse transcriptase, and 5 μl 1× yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37C for two hr. The reaction mixture is then incubated for 20 min at 85C, and probes are purified using two successive CHROMA SPIN+TE 30 columns (Clontech Laboratories). Purified probe is ethanol precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800×g, and the pellet is resuspended in 12 μl resuspension buffer, heated to 65C for five min, and mixed thoroughly. The probe is heated and mixed as before and then stored on ice. Probe is used in high density array-based hybridizations as described below.

Membrane-based Hybridization

Membranes are pre-hybridized in hybridization solution containing 1% Sarkosyl and 1× high phosphate buffer (0.5 M NaCl, 0.1 M Na₂HPO₄, 5 mM EDTA, pH 7) at 55C for two hr. The probe, diluted in 15 ml fresh hybridization solution, is then added to the membrane. The membrane is hybridized with the probe at 55C for 16 hr. Following hybridization, the membrane is washed for 15 min at 25C in 1 mM Tris (pH 8.0), 1% Sarkosyl, and four times for 15 min each at 25C in 1 mM Tris (pH 8.0). To detect hybridization complexes, XOMAT-AR film (Eastman Kodak, Rochester NY) is exposed to the membrane overnight at −70C, developed, and examined visually.

Polymer Coated Slide-based Hybridization

The following method was used in the microarray analysis presented in Table 3. Probe is heated to 65C for five min, centrifuged five min at 9400 rpm in a 5415C microcentrifuge (Eppendorf Scientific, Westbury N.Y.), and then 18 μl is aliquoted onto the array surface and covered with a coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hr at 60C. The arrays are washed for 10 min at 45C in 1×SSC, 0.1% SDS, and three times for 10 min each at 45C in 0.1=SSC, and dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of genes in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to equal numbers of probes derived from both biological samples give a distinct combined fluorescence (Shalon WO95/35505).

Hybridization complexes are detected with a microscope equipped with an Innova 70 mixed gas 10 W laser (Coherent, Santa Clara Calif.) capable of generating spectral lines at 488 nm for excitation of Cy3 and at 632 nm for excitation of Cy5. The excitation laser light is focused on the array using a 20× microscope objective (Nikon, Melville N.Y.). The slide containing the array is placed on a computer-controled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Filters positioned between the array and the photomultiplier tubes are used to separate the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomics).

OPCR Analysis

For QPCR, cDNA was synthesized from 1 ug total RNA in a 25 ul reaction with 100 units M-MLV reverse transcriptase (Ambion, Austin Tex.), 0.5 mM dNTPs (Epicentre, Madison Wis.), and 40 ng/ml random hexamers (Fisher Scientific, Chicago Ill.). Reactions were incubated at 25C for 10 minutes, 42C for 50 minutes, and 70C for 15 minutes, diluted to 500 ul, and stored at −30C. PCR primers and probes (5′6-FAM-labeled, 3′ TAMRA) were designed using PRIMER EXPRESS 1.5 software (ABI) and synthesized by ABI or Biosearch Technologies (Novato Calif.).

QPCR reactions were performed using an PRISM 7700 sequence detection system (ABI) in 25 ul total volume with 5 ul cDNA template, 1× TAQMAN UNIVERSAL PCR master mix (ABI), 100 nM each PCR primer, 200 nM probe, and 1× VIC-labeled beta-2-microglobulin endogenous control (ABI). Reactions were incubated at 50C for 2 minutes, 95C for 10 minutes, followed by 40 cycles of incubation at 95C for 15 seconds and 60C for 1 minute. Emissions were measured once every cycle, and results were analyzed using SEQUENCE DETECTOR 1.7 software (ABI) and relative expression, relative concentration of mRNA as compared to standards, were calculated using the comparative CT method (ABI User Bulletin #2). This method was used to produce the data for FIGS. 3 and 4.

VIII Complementary Molecules

Molecules complementary to the cDNA, from about 5 (PNA) to about 5000 bp (complement of a cDNA insert), are used to detect or inhibit gene expression. Detection is described in Example VII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in “triple helix” base pairing to compromise the ability of the double helix to open sufficiently for the binding of polymerases, transcription factors, or regulatory molecules. To inhibit translation, a complementary molecule is designed to prevent ribosomal binding to the mRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if elements for inducing vector replication are used in the transformation/expression system.

Stable transformation of dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the cDNA encoding the protein.

IX Protein Expression and Purification

Expression and purification of the protein are achieved using either a mammalian cell expression system or an insect cell expression system. The pUB6/V5-His vector system (Invitrogen) is used to express LCAP in CHO cells. The vector contains the selectable bsd gene, multiple cloning sites, the promoter/enhancer sequence from the human ubiquitin C gene, a C-terminal V5 epitope for antibody detection with anti-V5 antibodies, and a C-terminal polyhistidine (6xHis) sequence for rapid purification on PROBOND resin (Invitrogen). Transformed cells are selected on media containing blasticidin.

Spodoptera frugiperda (Sf9) insect cells are infected with recombinant Autographica californica nuclear polyhedrosis virus (baculovirus). The polyhedrin gene is replaced with the cDNA by homologous recombination and the polyhedrin promoter drives cDNA transcription. The protein is synthesized as a fusion protein with 6xhis which enables purification as described above. Purified protein is used in the following activity and to make antibodies

X Production of Specific Antibodies

Purification using polyacrylamide gel electrophoresis or similar techniques is used to isolate protein for immunization of hosts or host cells to produce antibodies using standard protocols.

Alternatively, the amino acid sequence of the protein is analyzed using readily available commercial software to determine regions of high immunogenicity. A peptide with high immunogenicity is cleaved, recombinantly-produced, or synthesized and used to raise antibodies by means known to those of skill in the art. Methods for selection of appropriate antigenic determinants such as those near the C-terminus or in hydrophilic regions are well described in the art (Ausubel (1989) supra, Ch. 11). Oligopeptides of about 15 residues in length are synthesized using an 431A peptide synthesizer (ABI) using FMOC chemistry and coupled to carriers such as BSA, thyroglobulin, or KLH (Sigma-Aldrich) by reaction with N-maleimidobenzoyl-N-hydroxysuccinimide ester to increase immunogenicity. The coupled peptide is then used to immunize the host. Rabbits are immunized with the oligopeptide-KLH complex in complete Freund's adjuvant. Resulting antisera are tested for antipeptide activity by binding the peptide to a substrate, blocking with 1% BSA, reacting with rabbit antisera, washing, and reacting with radio-iodinated goat anti-rabbit IgG.

XI Immunopurification Using Antibodies

Naturally occurring or recombinantly produced protein is purified by immunoaffinity chromatography using antibodies which specifically bind the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the purified protein is collected.

XII Antibody Arrays

Protein:Protein Interactions

In an alternative to yeast two hybrid system analysis of proteins, an antibody array can be used to study protein-protein interactions and phosphorylation. A variety of protein ligands are immobilized on a membrane using methods well known in the art. The array is incubated in the presence of cell lysate until protein:antibody complexes are formed. Proteins of interest are identified by exposing the membrane to an antibody specific to the protein of interest. In the alternative, a protein of interest is labeled with digoxigenin (DIG) and exposed to the membrane; then the membrane is exposed to anti-DIG antibody which reveals where the protein of interest forms a complex. The identity of the proteins with which the protein of interest interacts is determined by the position of the protein of interest on the membrane.

Proteomic Profiles

Antibody arrays can also be used for high-throughput screening of recombinant antibodies. Bacteria containing antibody genes are robotically-picked and gridded at high density (up to 18,342 different double-spotted clones) on a filter. Up to 15 antigens at a time are used to screen for clones to identify those that express binding antibody fragments. These antibody arrays can also be used to identify proteins which are differentially expressed in samples (de Wildt, supra)

XIII Screening Molecules for Specific Binding with the cDNA or Protein

The cDNA, or fragments thereof, or the protein, or portions thereof, are labeled with ³²P-dCTP, Cy3-dCTP, or Cy5-dCTP (APB), or with BIODIPY or FITC (Molecular Probes), respectively. Libraries of candidate molecules or compounds previously arranged on a substrate are incubated in the presence of labeled cDNA or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XIV Two-Hybrid Screen

A yeast two-hybrid system, MATCHMAKER LexA Two-Hybrid system (Clontech Laboratories), is used to screen for peptides that bind the protein of the invention. A cDNA encoding the protein is inserted into the multiple cloning site of a pLexA vector, ligated, and transformed into E. coli. cDNA, prepared from mRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ] cells using a polyethylene glycol/lithium acetate protocol. Transformed yeast cells are plated on synthetic dropout (SD) media lacking histidine (-His), tryptophan (-Trp), and uracil (-Ura), and incubated at 30C until the colonies have grown up and are counted. The colonies are pooled in a minimal volume of 1× TE (pH 7.5), replated on SD/-His/-Leu/-Trp/-Ura media supplemented with 2% galactose (Gal), 1% raffinose (Raf), and 80 mg/ml 5-bromo-4-chloro-3-indolyl β-d-galactopyranoside (X-Gal), and subsequently examined for growth of blue colonies. Interaction between expressed protein and cDNA fusion proteins activates expression of a LEU2 reporter gene in EGY48 and produces colony growth on media lacking leucine (-Leu). Interaction also activates expression of β-galactosidase from the p8op-lacZ reporter construct that produces blue color in colonies grown on X-Gal.

Positive interactions between expressed protein and cDNA fusion proteins are verified by isolating individual positive colonies and growing them in SD/-Trp/-Ura liquid medium for 1 to 2 days at 30C. A sample of the culture is plated on SD/-Trp/-Ura media and incubated at 30C until colonies appear. The sample is replica-plated on SD/-Trp/-Ura and SD/-His/-Trp/-Ura plates. Colonies that grow on SD containing histidine but not on media lacking histidine have lost the pLexA plasmid. Histidine-requiring colonies are grown on SD/Gal/Raf/X-Gal/-Trp/-Ura, and white colonies are isolated and propagated. The pB42AD-cDNA plasmid, which contains a cDNA encoding a protein that physically interacts with the protein, is isolated from the yeast cells and characterized.

XV Protease Assay

The protease activity and specificity of the cathepsin encoded by SEQ ID NO:2 is based on the rate of cleavage of specific peptide substrates and determination of an inhibitor profile. Rates of cleavage for LCAP are assessed by incubation of the protease with substrates such as Z-Phe-Arg-AMC or Bz-Val-Lys-Lys-Arg-AFC and by measuring the rate of release of the fluorescent or chromogenic leaving groups. Further specificity of the protease can be examined by titrating specific inhibitors into the cleavage assays and examining the change in the rate of proteolysis. Inhibitors for cathepsin L include trans-epoxysuccinyl-L-leucylamido-(3-methyl)butane, trans-epoxysuccinyl-L-leucylamido-(4-guanidino)butane, and chymostatin.

All patents and publications mentioned in the specification are incorporated by reference herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

What is claimed is:

1. A purified protein comprising a polypeptide having an amino acid sequence of SEQ ID NO:1.

2. A biologically active portion of the protein of claim 1 selected from about residue L114 to about residue S333; from about residue Q132 to about residue A143, from about residue L275 to about residue G285, and from about residue Y296 to about residue K314 of SEQ ID NO:1.

3. An antigenic determinant of the protein of claim 1 selected from about residue L280 to about residue K295 and from about residue A17 to about residue W32 of SEQ ID NO:1

4. A composition comprising the protein of claim 1 and a labeling moiety.

5. A composition comprising the protein of claim 1 and a pharmaceutical carrier.

6. An array element comprising the protein of claim 1.

7. A substrate upon which the protein of claim 1 is immobilized.

8. A method for using a protein to diagnose a cancer comprising:
a) performing an assay to quantify the expression of the protein of claim 1 in a sample;
b) comparing the expression of the protein to standards, thereby diagnosing cancer.

9. The method of claim 8 wherein the sample is selected from bladder, breast, colon, kidney, lung, lymph node, ovary and uterus.

10. The method of claim 8 wherein the expression is diagnostic of lung cancer.

11. A method for using a protein to identify and purify an antibody that specifically binds the protein comprising:
a) contacting a plurality of antibodies with the protein of claim 1 under conditions to allow the formation of an antibody:protein complex, and
b) dissociating the antibody from the antibody:protein complex, thereby obtaining purified antibodies that specifically binds the protein.

12. The method of claim 11, wherein the plurality of antibodies are selected from a polyclonal antibody, a monoclonal antibody, a chimeric antibody, a recombinant antibody, a humanized antibody, a single chain antibody, a Fab fragment, an F(ab′)₂ fragment, an Fv fragment; and an antibody-peptide fusion protein.

13. A method of using a protein to prepare and purify a polyclonal antibody comprising:
a) immunizing a animal with an antigenic determinant of claim 3 under conditions to elicit an antibody response;
b) isolating animal antibodies;
c) attaching the protein to a substrate;
d) contacting the substrate with isolated antibodies under conditions to allow specific binding to the protein;
e) dissociating the antibodies from the protein, thereby obtaining purified polyclonal antibodies.

14. A method of using a protein to prepare a monoclonal antibody comprising:
a) immunizing a animal with an antigenic determinant of claim 3 under conditions to elicit an antibody response;
b) isolating antibody-producing cells from the animal;
c) fusing the antibody-producing cells with immortalized cells in culture to form monoclonal antibody producing hybridoma cells;
d) culturing the hybridoma cells; and
e) isolating monoclonal antibodies from culture.

15. A method for using a protein to screen a plurality of molecules and compounds to identify at least one ligand, the method comprising:
a) combining the protein of claim 1 with a plurality of molecules and compounds under conditions to allow specific binding; and
b) detecting specific binding, thereby identifying a ligand that specifically binds the protein.

16. The method of claim 15 wherein the plurality of molecules and compounds are selected from agonists, antagonists, DNA molecules, small drug molecules, inhibitors, mimetics, peptides, peptide nucleic acids, proteins, and RNA molecules.

17. A purified antibody identified by the method of claim 11.

18. A method for using an antibody to detect expression of a protein in a sample, the method comprising:
a) combining the antibody of claim 17 with a sample under conditions which allow the formation of antibody:protein complexes; and
b) detecting complex formation, wherein complex formation indicates expression of the protein in the sample.

19. The method of claim 18 wherein the sample is selected from bladder, breast, colon, kidney, lung, lymph node, ovary and uterus.

20. The method of claim 18 wherein complex formation is compared with standards and is diagnostic of lung cancer.

21. A method for using an antibody to immunopurify a protein comprising:
a) attaching the antibody of claim 17 to a substrate,
b) exposing the antibody to a sample containing protein under conditions to allow antibody:protein complexes to form,
c) dissociating the protein from the complex, and
d) collecting the purified protein.

22. A composition comprising an antibody of claim 17 and a labeling moiety.

23. An antagonist identified by the method of claim 15.

24. A small drug molecule identified by the method of claim 15.
